SEQUENCE LISTING (85 sequences)

SEQ ID NO: 1 - DNA, 360 bp, Mus musculus
  source 1..360: /organism=“Mus musculus” /mol_type=“unassigned DNA”
cagatccagttggtgcagtctggacctgagctgaagaagcctggagagacagtcaagatctccagcaaggcttctggttattccttcacagactcttcaattcactgggtgaagcaggctccaggaaagggtttaaagtggatgggctggataaacactgagactggtgagccaacatatgcagatgacttcaagggacggtttgtcttctctttggaaacctctggcagcactgcctatttgcagatcaacaacctcaaaaatgaggacacggccacatatttctgtgctagaggaggctactatgattacgcatggtttgcttactggggccaagggactctggtcactgtctctgca

SEQ ID NO: 2 - DNA, 331 bp, Mus musculus
  source 1..331: /organism=“Mus musculus” /mol_type=“unassigned DNA”
gacattgtactgacacagtctcctgcttccttaactgtatctctggggcagagggccaccatctcatgcagggccagcaaaagtgtcagtacatctggctatagttttattcactggtaccaacagaacccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctcccacgttcggtgctgggaccaagctggagctga

SEQ ID NO: 3 - protein, 120 aa, Mus musculus
QIQLVQSGPELKKPGETVKISSKASGYSFTDSSIHWVKQAPGKGLKWMGWINTETGEPTYADDFKGRFVFSLETSGSTAYLQINNLKNEDTATYFCARGGYYDYAWFAYWGQGTLVTVSA

SEQ ID NO: 4 - protein, 110 aa, Mus musculus
DIVLTQSPASLTVSLGQRATISCRASKSVSTSGYSFIHWYQQNPGQPPKLLIYLASNLESGVPARFSGSGSGTDFTLNIHPVEEEDAATYYCQHSRELPPTFGAGTKLEL

SEQ ID NO: 5 - protein, 5 aa, Mus musculus
DSSIH

SEQ ID NO: 6 - protein, 17 aa, Mus musculus
WINTETGEPTYADDFKG

SEQ ID NO: 7 - protein, 11 aa, Mus musculus
GGYYDYAWFAY

SEQ ID NO: 8 - protein, 15 aa, Mus musculus
RASKSVSTSGYSFIH

SEQ ID NO: 9 - protein, 7 aa, Mus musculus
LASNLES

SEQ ID NO: 10 - protein, 9 aa, Mus musculus
QHSRELPPT

SEQ ID NO: 11 - DNA, 360 bp, Mus musculus
  source 1..360: /organism=“Mus musculus” /mol_type=“unassigned DNA”
caggtccaactgcagcagcctggggctgagcttgtgaagcctgggacttcagtgaagctgtcctgcaaggcttctggctacaacttcaccagctactggataaactgggtgaagctgaggcctggacaaggccttgagtggattggagatatttatcctggtagtggtagtactaactacaatgagaagttcaagagcaaggccacactgactgtagacacatcctccagcacagcctacatgcaactcagcagcctggcatctgaggactctgctctctattactgtgcaagagggcactacggtagtagctacggctttgactactggggccaaggcaccactctcacagtctcctca

SEQ ID NO: 12 - DNA, 335 bp, Mus musculus
  source 1..335: /organism=“Mus musculus” /mol_type=“unassigned DNA”
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttcctctcacgttcggtgctgggaccaagctggagctgaaacg

SEQ ID NO: 13 - protein, 120 aa, Mus musculus
QVQLQQPGAELVKPGTSVKLSCKASGYNFTSYWINWVKLRPGQGLEWIGDIYPGSGSTNYNEKFKSKATLTVDTSSSTAYMQLSSLASEDSALYYCARGHYGSSYGFDYWGQGTTLTVSS

SEQ ID NO: 14 - protein, 111 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 15 - protein, 5 aa, Mus musculus
SYWIN

SEQ ID NO: 16 - protein, 17 aa, Mus musculus
DIYPGSGSTNYNEKFKS

SEQ ID NO: 17 - protein, 11 aa, Mus musculus
GHYGSSYGFDY

SEQ ID NO: 18 - protein, 15 aa, Mus musculus
RASKSVSTSGYSYMH

SEQ ID NO: 19 - protein, 7 aa, Mus musculus
LASNLES

SEQ ID NO: 20 - protein, 9 aa, Mus musculus
QHSRELPLT

SEQ ID NO: 21 - DNA, 342 bp, Mus musculus
  source 1..342: /organism=“Mus musculus” /mol_type=“unassigned DNA”
caggtccagctgcagcagtctgggcctgaggtggtgaggcctggggtctcagtgaagatttcctgcaagggttccggctacacattcactgattatgctatgcactgggtgaagcagagtcatgcaaagagtctagagtggattggagttattagtacttacaatggtaatacaaactacaaccagaagtttaagggcaaggccacaatgactgtagacaaatcctccagcacagcctatatggaacttgccagattgacatctgaggattctgccatctattactgtgcaagagactatcttgactactggggccaaggcaccactctcacagtctcctca

SEQ ID NO: 22 - DNA, 341 bp, Mus musculus
  source 1..341: /organism=“Mus musculus” /mol_type=“unassigned DNA”
gacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggaccaagctggaaataaaacg

SEQ ID NO: 23 - protein, 114 aa, Mus musculus
QVQLQQSGPEVVRPGVSVKISCKGSGYTFTDYAMHWVKQSHAKSLEWIGVISTYNGNTNYNQKFKGKATMTVDKSSSTAYMELARLTSEDSAIYYCARDYLDYWGQGTTLTVSS

SEQ ID NO: 24 - protein, 113 aa, Mus musculus
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 25 - protein, 5 aa, Mus musculus
DYAMH

SEQ ID NO: 26 - protein, 17 aa, Mus musculus
VISTYNGNTNYNQKFKG

SEQ ID NO: 27 - protein, 5 aa, Mus musculus
DYLDY

SEQ ID NO: 28 - protein, 17 aa, Mus musculus
KSSQSLLNSGNQKNYLT

SEQ ID NO: 29 - protein, 7 aa, Mus musculus
WASTRES

SEQ ID NO: 30 - protein, 9 aa, Mus musculus
QNDYSYPFT

SEQ ID NO: 31 - DNA, 351 bp, Mus musculus
  source 1..351: /organism=“Mus musculus” /mol_type=“unassigned DNA”
gatgttcagctgcagcagtctggggcagaccttgtgaagccaggggcctcagtcaagttgtcctgcacagcttctggcttcaacattaaagacgcctatatgcactgggtgattcagaggcctgaacagggcctagaatggattggaagaattgatcctgcgaatggtgatactgaatatgacccgaacttccagggcaaggcctctatcacagcagacacatcctccaacacagcctacctgcaggtcagcagcctgacatctgaggacactgccgtctattactgtgctagggactatggtaactactttgacttctggggccaaggcaccactctcacagtctcctca

SEQ ID NO: 32 - DNA, 334 bp, Mus musculus
  source 1..334: /organism=“Mus musculus” /mol_type=“unassigned DNA”
gacattgtgctgacccaatctccaggttctttggctgtgtctctagggcagagggccaccatctcctgcagagccagtgaaagtgttgataattatggcattagttttatgaactggttccaattgaaaccaggacagccacccaaactcctcatctatgctgcatccaaccaaggatccggggtccctgccaggtttagtggcagtgggtctgggacagacttcagcctcaacatccatcctatggaggaggatgatagtgcaatgtatttctgtcagcaaagtaaggaggttcctggacgttcggtggaggcaccaagctggaaatcaaacg

SEQ ID NO: 33 - protein, 117 aa, Mus musculus
DVQLQQSGADLVKPGASVKLSCTASGFNIKDAYMHWVIQRPEQGLEWIGRIDPANGDTEYDPNFQGKASITADTSSNTAYLQVSSLTSEDTAVYYCARDYGNYFDFWGQGTTLTVSS

SEQ ID NO: 34 - protein, 111 aa, Mus musculus
DIVLTQSPGSLAVSLGQRATISCRASESVDNYGISFMNWFQLKPGQPPKLLIYAASNQGSGVPARFSGSGSGTDFSLNIHPMEEDDSAMYFCQQSKEVPGRSVEAPSWKSN

SEQ ID NO: 35 - protein, 5 aa, Mus musculus
DAYMH

SEQ ID NO: 36 - protein, 17 aa, Mus musculus
RIDPANGDTEYDPNFQG

SEQ ID NO: 37 - protein, 8 aa, Mus musculus
DYGNYFDF

SEQ ID NO: 38 - protein, 15 aa, Mus musculus
RASESVDNYGISFMN

SEQ ID NO: 39 - protein, 7 aa, Mus musculus
AASNQGS

SEQ ID NO: 40 - protein, 9 aa, Mus musculus
QQSKEVPGR

SEQ ID NO: 41 - DNA, 363 bp, Mus musculus
  source 1..363: /organism=“Mus musculus” /mol_type=“unassigned DNA”
cagatccagttggtgcagtctggacctgagctgaagaagcctggagagacagtcaggatctcctgcaaggcttctgggtataccttcacaaactatggaatgaactgggtgaagcaggctccaggaaagggtttaaagtggatgggctggataaacaccaacactggagagccaacatatgctgaagagttcaagggacggtttgccttctctttggaaacctctgccagcactgcctatttgcagatcaacaacctcaaaaatgaggacacggctacatatttctgtgcaagaggggggagatacggtaggggctggtacttcgatatctggggcgcagggaccacggtcaccgtctcctca

SEQ ID NO: 42 - DNA, 323 bp, Mus musculus
  source 1..323: /organism=“Mus musculus” /mol_type=“unassigned DNA”
caaattgttctcacccagtctccagcaatcatgtctgcatctccaggggagaaggtcaccatgacctgcagtgccagctcaagtgtaagttacatgtactggttccagcagaagccaggatcctcccccagactcctgatttatgacacatccaacctggcttctggagtccctcttcgcttcagtggcagtgggtctgggacctcttactctctcacaatcagccgaatggaggctgaagatgctgccacttattactgccagcagtggagtagttacccacccatcacgttcggaggggggaccaagttggaaataaaacg

SEQ ID NO: 43 - protein, 121 aa, Mus musculus
QIQLVQSGPELKKPGETVRISCKASGYTFTNYGMNWVKQAPGKGLKWMGWINTNTGEPTYAEEFKGRFAFSLETSASTAYLQINNLKNEDTATYFCARGGRYGRGWYFDIWGAGTTVTVSS

SEQ ID NO: 44 - protein, 107 aa, Mus musculus
QIVLTQSPAIMSASPGEKVTMTCSASSSVSYMYWFQQKPGSSPRLLIYDTSNLASGVPLRFSGSGSGTSYSLTISRMEAEDAATYYCQQWSSYPPITFGGGTKLEIK

SEQ ID NO: 45 - protein, 5 aa, Mus musculus
NYGMN

SEQ ID NO: 46 - protein, 17 aa, Mus musculus
WINTNTGEPTYAEEFKG

SEQ ID NO: 47 - protein, 12 aa, Mus musculus
GGRYGRGWYFDI

SEQ ID NO: 48 - protein, 10 aa, Mus musculus
SASSSVSYMY

SEQ ID NO: 49 - protein, 7 aa, Mus musculus
DTSNLAS

SEQ ID NO: 50 - protein, 10 aa, Mus musculus
QQWSSYPPIT

SEQ ID NO: 51 - DNA, 345 bp, Mus musculus
  source 1..345: /organism=“Mus musculus” /mol_type=“unassigned DNA”
caggtccagctgcagcagtctggagatgatctggtaaagcctggggcctcagtgaagctgtcctgcaaggcttctggctacaccttcaccagctactggattaactggataaaacagaggcctggacagggccttgagtggataggacgaattgctcctggaagtggtagtacttactacagtaaaatattcaagggcaaggcaacactgactgtagacacatcctccagcacagcctacattcagctcagcagcctgtcatctgaggactctgctgtctatttctgttcaagaggagattacggggactactggggccaaggcaccactctcacagtctcctca

SEQ ID NO: 52 - DNA, 320 bp, Mus musculus
  source 1..320: /organism=“Mus musculus” /mol_type=“unassigned DNA”
gacattgtgatgacccagtctcacaaattcatgtccacatcagtaggagacagggtcagcatcacctgcaaggccagtcaggatgtgggtactgctgtagcctggtatcaacagaaaccaggggaatctcctaaactactgatttactgggcatccacccggcacactggagtccctgatcgcctcacaggcagtggatctgggacagatttcactctcaccattagcaatgtgcactctgaagacttggcagattatttctgtcagcaatatagcggctatccgctcacgttcggtgatgggaccaagctggagctgaa

SEQ ID NO: 53 - protein, 115 aa, Mus musculus
QVQLQQSGDDLVKPGASVKLSCKASGYTFTSYWINWIKQRPGQGLEWIGRIAPGSGSTYYSKIFKGKATLTVDTSSSTAYIQLSSLSSEDSAVYFCSRGDYGDYWGQGTTLTVSS

SEQ ID NO: 54 - protein, 106 aa, Mus musculus
DIVMTQSHKFMSTSVGDRVSITCKASQDVGTAVAWYQQKPGESPKLLIYWASTRHTGVPDRLTGSGSGTDFTLTISNVHSEDLADYFCQQYSGYPLTFGDGTKLEL

SEQ ID NO: 55 - protein, 5 aa, Mus musculus
SYWIN

SEQ ID NO: 56 - protein, 17 aa, Mus musculus
RIAPGSGSTYYSKIFKG

SEQ ID NO: 57 - protein, 6 aa, Mus musculus
GDYGDY

SEQ ID NO: 58 - protein, 11 aa, Mus musculus
KASQDVGTAVA

SEQ ID NO: 59 - protein, 7 aa, Mus musculus
WASTRHT

SEQ ID NO: 60 - protein, 9 aa, Mus musculus
QQYSGYPLT

SEQ ID NO: 61 - DNA, 366 bp, Mus musculus
  source 1..366: /organism=“Mus musculus” /mol_type=“unassigned DNA”
caggtgcagctgaaggagtcaggacctggcctggtggcgccctcacagagcctgtccatcacatgcactgtctcagggttctcattaaccagctatggtgtaagctgggttcgccagcctccaggaaagggtctggagtggctgggagttatatggggtgacgggaccacaaattatcattcagctctcatatccagactgagcatcagcaaggataactccaagagccaagttttcttaaaactgaacagtctgcaaattgatgacacagccacgtattactgtgccaaaccgggggattactacggtagtagctactggtacttcgatgtctggggcgcagggaccacggtcaccgtctcctca

SEQ ID NO: 62 - DNA, 338 bp, Mus musculus
  source 1..338: /organism=“Mus musculus” /mol_type=“unassigned DNA”
gatgttgttctgacccaaactccactctctctgcctgtcaatattggagatcaagcctctatctcttgcaagtctactaagagtcttctgaatagtgatggattcacttatttggactggtacctgcagaagccaggccagtctccacagctcctaatatatttcgtttctaatcgattttctggagttccagacaggttcagtggcagtgggtcaggaacagatttcacactcaagatcagcagagtggaggctgacgatttgggagtttattattgcttccagagtaactatcttccgtacacgttcggaggggggaccaagctggaaataaaacg

SEQ ID NO: 63 - protein, 122 aa, Mus musculus
QVQLKESGPGLVAPSQSLSITCTVSGFSLTSYGVSWVRQPPGKGLEWLGVIWGDGTTNYHSALISRLSISKDNSKSQVFLKLNSLQIDDTATYYCAKPGDYYGSSYWYFDVWGAGTTVTVSS

SEQ ID NO: 64 - protein, 112 aa, Mus musculus
DVVLTQTPLSLPVNIGDQASISCKSTKSLLNSDGFTYLDWYLQKPGQSPQLLIYFVSNRFSGVPDRFSGSGSGTDFTLKISRVEADDLGVYYCFQSNYLPYTFGGGTKLEIK

SEQ ID NO: 65 - protein, 5 aa, Mus musculus
SYGVS

SEQ ID NO: 66 - protein, 16 aa, Mus musculus
VIWGDGTTNYHSALIS

SEQ ID NO: 67 - protein, 14 aa, Mus musculus
PGDYYGSSYWYFDV

SEQ ID NO: 68 - protein, 16 aa, Mus musculus
KSTKSLLNSDGFTYLD

SEQ ID NO: 69 - protein, 7 aa, Mus musculus
FVSNRFS

SEQ ID NO: 70 - protein, 9 aa, Mus musculus
FQSNYLPYT

SEQ ID NO: 71 - DNA, 363 bp, Mus musculus
  source 1..363: /organism=“Mus musculus” /mol_type=“unassigned DNA”
gaggtccagctgcagcagtctggacctgagctagtgaagactggggcttcagtgaagatatcctgcaaggcttctggttactcattcactggttactacatgcactgggtcaagcagagccatggaaagagccttgagtggattggatatattagttgttacaatggtgctactagctacaaccagaagttcaagggcaaggccacatttactgtagacacatcctccagcacagcctacatgcagttcaacagcctgacatctgaagactctgcggtctattactgtgcaagaggaggtaactgggacgtggagtatgctatggactactggggtcaaggaacctcagtcaccgtctcctca

SEQ ID NO: 72 - DNA, 335 bp, Mus musculus
  source 1..335: /organism=“Mus musculus” /mol_type=“unassigned DNA”
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccattcacgttcggctcggggacaaagttggaaataaaacg

SEQ ID NO: 73 - protein, 121 aa, Mus musculus
EVQLQQSGPELVKTGASVKISCKASGYSFTGYYMHWVKQSHGKSLEWIGYISCYNGATSYNQKFKGKATFTVDTSSSTAYMQFNSLTSEDSAVYYCARGGNWDVEYAMDYWGQGTSVTVSS

SEQ ID NO: 74 - protein, 111 aa, Mus musculus
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPFTFGSGTKLEIK

SEQ ID NO: 75 - protein, 5 aa, Mus musculus
GYYMH

SEQ ID NO: 76 - protein, 17 aa, Mus musculus
YISCYNGATSYNQKFKG

SEQ ID NO: 77 - protein, 12 aa, Mus musculus
GGNWDVEYAMDY

SEQ ID NO: 78 - protein, 15 aa, Mus musculus
RASKSVSTSGYSYMH

SEQ ID NO: 79 - protein, 7 aa, Mus musculus
LASNLES

SEQ ID NO: 80 - protein, 9 aa, Mus musculus
QHSRELPFT

SEQ ID NO: 81 - DNA, 351 bp, Mus musculus
  source 1..351: /organism=“Mus musculus” /mol_type=“unassigned DNA”
cagggtcagatgcagcagtctggagctgagctggtgaagcctggggcttcagtgaagctgtcctgcaagacttctggcttcaccttcagcagtaactatataagttggttgaagcaaaagcctggacagagtcttgagtggattgcatggatttatgctggaactggtggtactaattataatcagaagttcacaggcaaggcccaactgactgtagacacatcctccagcacagcctacatacaattcagcagcctgacaactgaggactctgccatctattactgtgcaagactctatggttactactttgactactggggccaaggcaccactctcacagtctcctca

SEQ ID NO: 82 - protein, 117 aa, Mus musculus
QGQMQQSGAELVKPGASVKLSCKTSGFTFSSNYISWLKQKPGQSLEWIAWIYAGTGGTNYNQKFTGKAQLTVDTSSSTAYIQFSSLTTEDSAIYYCARLYGYYFDYWGQGTTLTVSS

SEQ ID NO: 83 - protein, 5 aa, Mus musculus
SNYIS

SEQ ID NO: 84 - protein, 17 aa, Mus musculus
WIYAGTGGTNYNQKFTG

SEQ ID NO: 85 - protein, 8 aa, Mus musculus
LYGYYFDY

(19) United States
(12) Patent Application Publication
(10) Pub. No.: US 2016/0194402 A1
(43) Pub. Date: Jul. 7, 2016

(54) CD70-BINDING PEPTIDES AND METHOD, PROCESS AND USE RELATING THERETO

(71) Applicant: ADURO BIOTECH HOLDINGS, EUROPE B.V., Oss (NL)

(72) Inventors: Hans VAN EENENNAAM, Nijmegen (NL); Andrea VAN ELSAS, Oss (NL); Lilian DRIESSEN, Baarlo (NL); Jannie BORST, Amsterdam (NL)

(21) Appl. No.: 15/061,445

(22) Filed: Mar. 4, 2016

Related U.S. Application Data
(63) Continuation-in-part of application No. PCT/EP2014/068960, filed on Sep. 5, 2014.

(30) Foreign Application Priority Data
Sep. 5, 2013 (NL) 2011389

(51) Int. Cl. C07K 16/28; G01N 33/566

(52) U.S. Cl. CPC C07K 16/2875; G01N 33/566; C07K 2317/24; C07K 2317/92; C07K 2317/73; G01N 2333/70575

(57) ABSTRACT

The invention relates to a method for obtaining CD70-binding peptides. With this method CD70-binding peptides may be obtained and/or selected. Further aspects of the invention relates to a cell comprising a nucleotide sequence coding for a CD70-binding peptide according to the invention, a process for producing a CD70-binding peptide and the CD70-binding peptide obtainable in the production process and/or the selection method. In view of the possible utility of the CD70-binding peptides according to the invention, further aspects of the invention relate to medical and diagnostic uses of a CD70-binding peptide of the invention.

### RELATED APPLICATIONS AND INCORPORATION BY REFERENCE

This application is a continuation-in-part application of international patent application Serial No. PCT/EP2014/068960 filed 5 Sep. 2014, which published as PCT Publication No. WO 2015/032906 on 12 Mar. 2015, which claims benefit of NL patent application Serial No. 2011389 filed 5 Sep. 2013.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

The instant application contains a Sequence Listing which has been submitted electronically in ASCII format and is hereby incorporated by reference in its entirety. Said ASCII copy, created on Mar. 4, 2016, is named 45468_00_2003_SL.txt and is 36,245 bytes in size.

### FIELD OF THE INVENTION

The present invention relates to the field of human and veterinary medicine, including medical/veterinary diagnosis and medical/veterinary research. More specifically the present invention relates to peptides, including antibodies, binding to cell-surface proteins, in particular CD70, suitable for use in this or other fields.

### BACKGROUND OF THE INVENTION

CD27, a TNF receptor family member was identified as a membrane molecule on human T cells (van Lier et al., 1987, J Immunol 139:1589-96). According to current evidence, CD27 has a single ligand, CD70, which is also a TNF family member (Goodwin et al., 1993, Cell 73:447-56).

CD27 is exclusively expressed by hematopoietic cells, in particular those of the lymphocyte lineage, i.e. T-, B- and NK cells. CD27 was originally defined as a human T-cell co-stimulatory molecule that increments the proliferative response to TCR stimulation (van Lier et al., 1987, J Immunol 139:1589-96). Presence of CD70 dictates the timing and persistence of CD27-mediated co-stimulation. Studies with CD70 blocking antibody in mouse models support the concept that CD27-CD70 interactions inhibit the activation of CD4⁺ and CD8⁺ effector T cells, e.g. after protein immunization, virus infection and allotransplantation (Taraban et al., 2004, J Immunol 173:6542-46; Bullock and Yagita, 2005, J Immunol 174:710-17; Yamada et al., 2005, J Immunol 174:1357-1364; Schildknecht et al., 2007, Eur J Immunol 37:716-28). Transgenic expression of CD70 in immature dendritic cells sufficed to convert immunological tolerance to virus or tumors into CD8⁺ T cell responsiveness. Likewise, agonistic soluble CD70 promoted the CD8⁺ T cell response upon such peptide immunization (Rowley et al., 2004, J Immunol 172:6039-6046) and in CD70 transgenic mice, CD4⁺ and CD8⁺ effector cell formation in response to TCR stimulation was greatly facilitated (Arens et al. 2001, Immunity 15:801-12; Tesselaar et al., 2003, Nat Immunol 4:49-54; Keller et al. 2008, Immunity 29: 334-346). In mouse lymphoma models, tumor rejection was improved upon CD70 transgenesis or injection of an activating anti-mouse CD27 antibody (Arens et al., 2003, J Exp Med 199:1595-1605; French et al., 2007, Blood 109: 4810-15; Sakanishi and Yagita, 2010, Biochem. Biophys. Res. Comm. 393: 829-835; WO 2008/051424; WO 2012/004367).

In addition, CD70 was demonstrated to induce CD27-mediated NK-cell activity, resulting in the rejection of CD70⁺ tumor cells by immunocompetent mice (Takeda et al., 2000, J Immunol 164; 1741-1745; Aulwurm et al., 2006, Int J Cancer 118:1728-1735 Taraban et al., 2008, J Immunol 139:1589-96). CD27-mediated NK cell activation also promoted the generation of CD8⁺ anti-tumor immunity (Kelly et al., 2002, Nat Immunol 3:83-90).

Many reports have identified CD70 as a tumor antigen, which is overexpressed on many different tumor types of lymphoid origin, like 71% of diffuse large B-cell lymphomas, 33% of follicular lymphomas, 50% of B-cell lymphocytic leukemias, 25% of Burkitt and mantle cell lymphomas and 100% of Waldenström macroglobulinemia as well as the majority of Hodgkin disease Reed-Sternberg cells. On solid tumors, CD70 overexpression has been described on nasopharyngeal carcinoma, EBV-negative thymic carcinoma, astrocytoma, glioblastoma and renal cell carcinoma (McEarchern et al., 2007, Blood 109:1185-92). Targeting CD70 as a tumor antigen and at the same time blocking the CD27-CD70 interaction using either non-conjugated or conjugated antibodies has proven to be a successful strategy in different (mouse) model systems (Law et al., 2006, Cancer Res. 66: 2328-37; McEarchern et al., 2007, Blood 109:1185-92; Alley et al., 2008, Bioconjugate Chem 19: 759-65; Ho et al., 2008; WO 2004/104045; WO 2004/073656; WO 2005/077462; WO 2006/044643; WO 2006/113909; WO 2007/038637; WO 2008/074004; WO 2012/058460; WO 2012/123586).

Citation or identification of any document in this application is not an admission that such document is available as prior art to the present invention.

### SUMMARY OF THE INVENTION

The inventors of the present invention have found that the CD70 binding antibodies commercially available block the CD27-CD70 interaction and thereby do not exploit the immune rejection potential encased in the CD27-CD70 pathway. It is expected that this is a common feature of known CD70-binding antibodies and any other known CD70-binding peptides. Without wishing to be bound by any theory, it is hypothesized that this may be due to immunodominance (or binding dominance) of epitopes at or around the region of CD70 that binds to CD27, causing the known antibodies (and other binding peptides) to bind at locations that hinder the CD27-CD70 interaction. This deficit of known CD70-binding peptides, such as antibodies, is not appreciated in the art.

On the basis of their findings, the inventors of the present invention set out to develop methods to identify and obtain anti-CD70 antibodies having a reduced blocking of the CD27-CD70 interaction. In particular methods were designed and developed to select the rarely abundant B-cells that express the reduced-blocking antibodies from CD70 immunized mice. This method at present resulted in the identification of nine reduced-blocking anti-CD70 antibodies.

The specific methods developed can be exploited more broadly. Thus on the basis of the developed methods additional reduced-blocking CD70-binding antibodies and/or other reduced-blocking CD70-binding peptides may be identified and/or obtained. Administration of reduced-blocking anti-CD70 peptides, including antibodies, alone or in combination with other agents to a mammal, such as a human, can for example be used in the treatment and/or diagnosis of cancer and/or in medical research, including veterinary research.

The invention thus according to a first aspect relates to a method for obtaining CD70-binding peptides. With this method CD70-binding peptides may be obtained and/or selected. The method may comprise:
providing a library of binder peptides;
selecting CD70-binding peptides from the library, by means of affinity selection using a target peptide immobilized on a solid support, said target peptide which may comprise a CD27 binding region of CD70 and a number of CD70 epitopes.

The method according to the invention is characterized in that the target peptide is immobilized on the solid support in interaction with a peptide, the shielding peptide, which may comprise a CD70 binding region of CD27 or a CD70 binding region of a binding equivalent of CD27 capable of ligating CD70. By selecting CD70-binding peptides on the basis of their affinity with a target peptide having the above features in interaction with a shielding peptide having the above features, it is prevented that binding peptides interfering with the CD27-CD70 interaction are selected as the CD70 binding peptides. Thus CD70-binding peptides having a reduced blocking of the CD27-CD70 interaction can be obtained.

A further aspect of the invention relates to a CD70-binding peptide, including an immunoglobulin or a binding immunoglobulin fragment, obtainable with the method according to the invention. Yet a further aspect of the invention relates to a cell which may comprise a nucleotide sequence coding for a CD70-binding peptide according to the invention. Such a cell may be used for producing a CD70-binding peptide according to the invention. The invention thus also relates to a process for producing a CD70-binding peptide according to the invention with the use of a cell according to the invention and the CD70-binding peptide obtainable in the production process. In view of the possible utility of the CD70-binding peptide according to the invention further aspects of the invention relate to a CD70-binding peptide of the invention for use as a medicament, a pharmaceutical composition which may comprise a CD70-binding peptide of the invention and the use of a CD70-binding peptide of the invention as a diagnostic tool.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE SEQUENCES

The sequences presented in the sequence listing relate to the amino acid sequences and encoding DNA sequences of the V_{H} and V_{L} chains of nine immunoglobulins (hCD70.17, hCD70.21, hCD70.23, hCD70.27, hCD70.29, hCD70.32, hCD70.34, hCD70.36, hCD70.39) obtained with the method of the invention. In addition the amino acid sequences of the CDR regions of both the V_{H} and V_{L} chains of these immunoglobulins are presented. Table 1 below correlates the sequence IDs to their respective sequence.

TABLE 1

| SEQ ID NO: | Description |
|---|---|
| 1 | hCD70.17 heavy chain variable region (DNA) |
| 2 | hCD70.17 light chain variable region (DNA) |
| 3 | hCD70.17 heavy chain variable region (AA) |
| 4 | hCD70.17 light chain variable region (AA) |
| 5 | hCD70.17 heavy chain CDR1 (AA) |
| 6 | hCD70.17 heavy chain CDR2 (AA) |
| 7 | hCD70.17 heavy chain CDR3 (AA) |
| 8 | hCD70.17 light chain CDR1 (AA) |
| 9 | hCD70.17 light chain CDR2 (AA) |
| 10 | hCD70.17 light chain CDR3 (AA) |
| 11 | hCD70.21 heavy chain variable region (DNA) |
| 12 | hCD70.21 light chain variable region (DNA) |
| 13 | hCD70.21 heavy chain variable region (AA) |
| 14 | hCD70.21 light chain variable region (AA) |
| 15 | hCD70.21 heavy chain CDR1 (AA) |
| 16 | hCD70.21 heavy chain CDR2 (AA) |
| 17 | hCD70.21 heavy chain CDR3 (AA) |
| 18 | hCD70.21 light chain CDR1 (AA) |
| 19 | hCD70.21 light chain CDR2 (AA) |
| 20 | hCD70.21 light chain CDR3 (AA) |
| 21 | hCD70.23 heavy chain variable region (DNA) |
| 22 | hCD70.23 light chain variable region (DNA) |
| 23 | hCD70.23 heavy chain variable region (AA) |
| 24 | hCD70.23 light chain variable region (AA) |
| 25 | hCD70.23 heavy chain CDR1 (AA) |
| 26 | hCD70.23 heavy chain CDR2 (AA) |
| 27 | hCD70.23 heavy chain CDR3 (AA) |
| 28 | hCD70.23 light chain CDR1 (AA) |
| 29 | hCD70.23 light chain CDR2 (AA) |
| 30 | hCD70.23 light chain CDR3 (AA) |
| 31 | hCD70.27 heavy chain variable region (DNA) |
| 32 | hCD70.27 light chain variable region (DNA) |
| 33 | hCD70.27 heavy chain variable region (AA) |
| 34 | hCD70.27 light chain variable region (AA) |
| 35 | hCD70.27 heavy chain CDR1 (AA) |
| 36 | hCD70.27 heavy chain CDR2 (AA) |
| 37 | hCD70.27 heavy chain CDR3 (AA) |
| 38 | hCD70.27 light chain CDR1 (AA) |
| 39 | hCD70.27 light chain CDR2 (AA) |
| 40 | hCD70.27 light chain CDR3 (AA) |
| 41 | hCD70.29 heavy chain variable region (DNA) |
| 42 | hCD70.29 light chain variable region (DNA) |
| 43 | hCD70.29 heavy chain variable region (AA) |
| 44 | hCD70.29 light chain variable region (AA) |
| 45 | hCD70.29 heavy chain CDR1 (AA) |
| 46 | hCD70.29 heavy chain CDR2 (AA) |
| 47 | hCD70.29 heavy chain CDR3 (AA) |
| 48 | hCD70.29 light chain CDR1 (AA) |
| 49 | hCD70.29 light chain CDR2 (AA) |
| 50 | hCD70.29 light chain CDR3 (AA) |
| 51 | hCD70.32 heavy chain variable region (DNA) |
| 52 | hCD70.32 light chain variable region (DNA) |
| 53 | hCD70.32 heavy chain variable region (AA) |
| 54 | hCD70.32 light chain variable region (AA) |
| 55 | hCD70.32 heavy chain CDR1 (AA) |
| 56 | hCD70.32 heavy chain CDR2 (AA) |
| 57 | hCD70.32 heavy chain CDR3 (AA) |
| 58 | hCD70.32 light chain CDR1 (AA) |
| 59 | hCD70.32 light chain CDR2 (AA) |
| 60 | hCD70.32 light chain CDR3 (AA) |
| 61 | hCD70.34 heavy chain variable region (DNA) |
| 62 | hCD70.34 light chain variable region (DNA) |
| 63 | hCD70.34 heavy chain variable region (AA) |
| 64 | hCD70.34 light chain variable region (AA) |
| 65 | hCD70.34 heavy chain CDR1 (AA) |
| 66 | hCD70.34 heavy chain CDR2 (AA) |
| 67 | hCD70.34 heavy chain CDR3 (AA) |
| 68 | hCD70.34 light chain CDR1 (AA) |
| 69 | hCD70.34 light chain CDR2 (AA) |
| 70 | hCD70.34 light chain CDR3 (AA) |
| 71 | hCD70.36 heavy chain variable region (DNA) |
| 72 | hCD70.36 light chain variable region (DNA) |
| 73 | hCD70.36 heavy chain variable region (AA) |
| 74 | hCD70.36 light chain variable region (AA) |
| 75 | hCD70.36 heavy chain CDR1 (AA) |
| 76 | hCD70.36 heavy chain CDR2 (AA) |
| 77 | hCD70.36 heavy chain CDR3 (AA) |
| 78 | hCD70.36 light chain CDR1 (AA) |
| 79 | hCD70.36 light chain CDR2 (AA) |
| 80 | hCD70.36 light chain CDR3 (AA) |
| 81 | hCD70.39 heavy chain variable region (DNA) |
| 82 | hCD70.39 heavy chain variable region (AA) |
| 83 | hCD70.39 heavy chain CDR1 (AA) |
| 84 | hCD70.39 heavy chain CDR2 (AA) |
| 85 | hCD70.39 heavy chain CDR3 (AA) |

### BRIEF DESCRIPTION OF THE FIGURES

The following detailed description, given by way of example, but not intended to limit the invention solely to the specific embodiments described, may best be understood in conjunction with the accompanying drawings.

FIGS. 1A-B. Characterization of commercially available anti-hCD70 antibodies. FIG. 1A demonstrates the binding of commercially available anti-CD70 antibodies to stably transfected CHO-K1.hCD70 cells. IgG1 was used as a negative control. FIG. 1B shows the blockade of the CD27-CD70 interaction by the commercially available antibodies. IgG1 was used as a negative control.

FIG. 2. Selection strategy to identify reduced-blocking anti-CD70 antibodies. Streptavin magnetic DynaBeads were loaded with biotin-hCD8-hCD70 recombinant protein, which after extensive washing was allowed to bind recombinant Fc-hCD27.

FIGS. 3A-B. Reduced-blocking anti-CD70 antibodies. FIG. 3A demonstrates the binding of reduced blocking anti-hCD70 antibodies to stably transfected CHO-K1.hCD70 cells. IgG1 was used as a negative control. FIG. 3B shows the reduced blockade of the CD27-CD70 interaction by the reduced-blocking anti-hCD70 antibodies using a cell-based CHO-K1.hCD27 assay and recombinant hCD70 (CD70(h)-muCD8) fusion Protein. IgG1 was used as a negative control.

FIGS. 4A-B. Non-blocking anti-CD70 antibodies. FIG. 4A demonstrates the binding of non-blocking anti-hCD70 antibodies to stably transfected CHO-K1.hCD70 cells. IgG1 was used as a negative control. FIG. 4B shows the absence of blockade of the CD27-CD70 interaction by these non-blocking anti-hCD70 antibodies. IgG1 was used as a negative control.

FIGS. 5A-B. Reduced inhibition of T-cell proliferation. FIG. 5A demonstrates that reduced-blocking anti-hCD70 reduce inhibition of the CD70 mediated T-cell activation, as was analyzed by blast formation using flow cytometry. Mouse IgG1 was used as a negative control. 2F2 antibody was used as a positive control. FIG. 5B demonstrates that reduced-blocking anti-hCD70 reduce inhibition of the CD70 mediated T-cell proliferation, as was analyzed by CFSE dilution using flow cytometry. Mouse IgG1 was used as a negative control. 2F2 antibody was used as a positive control.

### DETAILED DESCRIPTION

In the method of the invention for obtaining CD70-binding peptides a library of binder peptides is provided. The term “library” is known within the art and within the known meaning of this term a “library of binder peptides” may be understood to mean a collection or array of differing binder peptides. The term “binder peptides” or alternatively “binding peptides” within the context of a peptide library may be understood as referring to peptides having a potential capability of binding other compounds and/or structures, in particular epitopes, more in particular peptidic epitopes. Within the present invention binder peptides in particular have a potential CD70-binding capability.

Antibodies (immunoglobulins) and binding fragments of antibodies, are known peptides having the potential capability to bind to other compounds and/or structures, including epitopes, such as peptidic epitopes. Thus within the present invention it is in particular envisaged to provide libraries of antibodies or antibody fragments. The skilled person will know how to obtain and thus how to provide a library of antibodies or antibody fragments.

Antibodies or antibody fragments may for example be isolated from antibody phage libraries generated using the techniques described in McCafferty et al., 1990, Nature, 348:552-554. Clackson et al., 1991, Nature, 352:624-628, and Marks et al., 1991, J. Mol. Biol. 222:581-597, who describe the isolation of murine and human antibodies, respectively, using phage libraries. Subsequent publications describe the production of high affinity (nM range) human antibodies by chain shuffling (Marks et al., 1992, Bio/Technology, 10:779-783), as well as combinatorial infection and in vivo recombination as a strategy for constructing very large phage libraries (Waterhouse et al., 1993, Nuc. Acids Res. 21:2265-2266).

Antibody or antibody fragments may be isolated from mRNA display libraries generated using techniques described in Fukuda et al., 2006, Nuc. Acids Res., 34:e127, who describe the isolation of antibody fragments using mRNA display libraries.

Alternatively an antibody library may comprise a collection of lymphocytes, preferably splenocytes, collected from a mammal, such as a non-human mammal, immunized with an agent suitable for eliciting a CD70-specific immune response in the mammal. Immunization of (non-human) mammals and collecting splenocytes (or other lymphocytes) is common practice within the field. The agent suitable for eliciting a CD70-specific immune response used for immunization may be the CD70 protein or a part thereof. Alternatively immunization may be effected by DNA immunization using a nucleotide sequence, preferably a cDNA sequence, coding for CD70 or a part thereof. Methods and procedures for DNA immune immunization are known to the skilled person. Exemplary procedures for DNA immunization are presented in the examples.

Apart from a library of antibodies (or antibody fragments), a library of binder peptides engineered on non-immunoglobulin protein scaffolds may be provided. Examples of such protein scaffolds include, but are not restricted to Adnectins, Affibodies, Anticalins and DARPins (Gebauer and Skerra, Current opinion Chem. Biol., 2009, 13:245-255 and Caravella and Lugovskoy, Current opinion Chem. Biol., 2010, 14:520-528). Selection methods for example include phage display to identify protein scaffolds that express CD70-binding peptides.

In addition, combinatorial peptide libraries may be provided as the binder peptide library. For example, one-bead-one-compound combinatorial libraries are libraries that express a broad set of peptides on beads, where one bead is binding one peptide. After selection procedures, beads are recovered and the peptide is identified (Lam et al., Methods, 1996, 9:482-93; Xiao et al., Comb. Chem. High Throughput Screen, 2013, Mar. 13 (epub ahead of print), for example using mass-spectrometry methods.

In the method for obtaining CD70-binding peptides, peptides binding specifically to CD70 are selected from the library of binder peptides by means of affinity selection, wherein the affinity selection procedure uses a target peptide immobilized on a solid support. “Specifically” binds, when referring to a ligand/receptor, antibody/antigen, or other binding pair, indicates a binding reaction which is determinative of the presence of the protein, e.g., CD70, in a heterogeneous population of proteins and/or other biologics. Thus, under designated conditions, a specified ligand/antigen binds to a particular receptor/antibody and does not bind in a significant amount to other proteins present in the sample. The target peptide may comprise a CD27-binding region of CD70 and a number of CD70 epitopes. Affinity selection procedures using an immobilized ligand for a binder peptide to be selected are known in the art. For example panning or biopanning procedures are known. As is known and as will be clear for the skilled person, a typical affinity selection procedure may comprise three steps: capturing, washing and identification of captured binders.

For the affinity selection procedure employed in the method of the present invention, the capturing step involves binding of the binder peptides of the library with a target peptide which may comprise a CD27-binding region of CD70. The target peptide is immobilized on a solid support to allow identification and/or isolation of binder peptides specifically interacting with the selected target. The term “immobilized” should be understood as meaning having a restricted, or reduced mobility. The restricted, or reduce mobility is relative to the washing medium, used in the washing step. The “immobilized” target peptide need not be directly bound to or interacting with the solid support. Instead it may have an interaction with a compound or moiety bound to or interacting with the solid support. Examples of immobilization of target peptide to the solid support include, but are not restricted to non-specific adherence to a solid support, such as plastic, NH₂-coupling to beads, binding to tosyl-activated beads or binding to Protein A beads. Such and other methods will be clear to the skilled person.

The target in the affinity selection procedure employed in the method of the invention may comprise a CD27-binding region of CD70 and a number of CD70 epitopes. The CD27-binding region of CD70 may be presented in the form of a complete CD70 protein or a part of a CD70 protein. The sequence of the CD70 protein or a part thereof preferably is of human origin. The CD70 epitopes may be present on the CD27-binding region of CD70 or on a different part of the target peptide. The selection of the CD27-binding region of CD70 and the CD70 epitopes is such that binding interaction of the target peptide with the shielding peptide is possible. In this description and the appended claims a number of should be understood as meaning one or more, such as 1, 2, 3, 4, 5, 6, 7 or more, every time when used, unless specifically stated differently.

In the method of the invention for obtaining CD70-binding peptides the target peptide (which may comprise a CD27-binding region of CD70 and a number of CD70 epitopes) is immobilized on the solid support in interaction with a shielding peptide which may comprise a CD70 binding region of CD27 or a CD70 binding region of a binding equivalent of CD27 capable of ligating CD70. The CD70-binding region of CD27 may be presented in a complete CD27 protein or in a part of a CD27 protein. The sequence of the CD27 protein or a part thereof preferably is of human origin.

As an alternative for the CD70 binding region of CD27, a CD70 binding peptide, which binds to the same region of CD70 as CD27 does, may be used. Such a binding equivalent of CD27 is capable of ligating (binding) CD70 and may for example be a peptide, such as an antibody, binding to CD70 at the CD70-CD27 binding interface. Such peptides are thus equivalent to CD27 in respect of its binding to (or ligation of) CD70. Thus while binding to CD70, the binding equivalent of CD27 will interfere with the interaction of CD27 with CD70. A binding equivalent of CD27 may thus be selected from peptides interfering with the interaction of CD70 with CD27. Such CD70 ligating CD27 binding equivalent peptides according to some embodiments may for example be selected from the antibodies 2F2 (CLB70/2; available from Pelicluster), Ki-24 (available from BD), DS-MB03194 (available from Ray Biotech), 10B1934 (available from US Biologicals), CM204154 (available from Int. Lab), BU69 (available from Santa Cruz), 7H173 (available from Life Span Biosciences). Whether or not a certain CD70-binding peptide, such as a CD70-binding antibody, interferes with the interaction of CD27 with CD70 may be determined in accordance with the methodology described in example 1 or 3.

Binding equivalent peptides capable of ligating CD70 which are suitable for use in the present invention may have an EC50 for CD70 binding of below 1·10⁻⁶ M, such as below 1·10⁻⁷ M, preferably between 1·10⁻⁶ to 1·10⁻¹¹ M, such as 1·10⁻⁷ to 1·10⁻¹¹ M. Alternatively or in conjuction the CD70 ligating CD27 binding equivalent peptides suitable for use in the present invention may for example have an IC50 for inhibition of CD70 binding to CD27 of between 1.5·10⁻⁸ to 1·10⁻¹¹ M, such as 4·10⁻⁹ to 1·10⁻¹¹ M, preferably 3·10⁻⁹ to 1·10⁻¹⁰ M, more preferably 2·10⁻⁹ to 5·10⁻¹⁰ M. The EC50 and/or IC50 of the CD70 ligating CD27 binding equivalent peptides may be determined with any suitable method known to the skilled person, in particular the methods described in example 1.

It should be noted that the target peptide may be immobilized on the solid support by its interaction with the shielding peptide, said shielding peptide being immobilized on the solid support by known means exemplified above.

The washing step follows the capturing step. In this step unbound elements (e.g. binder peptides and/or target peptides and/or shielding peptides and/or other elements) are washed from the solid support by use of a washing medium, such as a washing liquid. By selecting the washing conditions the stringency of the selection may be selected. Such procedures are clear to a skilled person. For example, washing procedures including cells will use Phosphate buffered saline or culture medium as a washing liquid. Washing liquid can include high salt (e.g. 1 M Sodium Chloride) or low salt (e.g. 50 mM Sodium Chloride) to influence the stringency (ionic strength) of washing procedures. Washing liquid can also include detergents, such as Nonidet P-40 to influence the stringency (hydrophobic strength) of washing procedures.

In the identification step following the washing step, binder peptides that remain in interaction with the target peptide after the washing step are identified. The identification step may comprise elution of binder peptides from the solid support where after the eluted binder peptides may be identified in any suitable way known. A skilled person can apply mass-spectrometry methods to identify peptides, RNA sequencing to identify RNA molecules encoding the binder peptide or DNA sequencing to identify cDNA molecules encoding the binder peptide. Alternatively identification may be done by using a labeling moiety, such as a fluorescent label, linked to either the binder peptides or the target peptide, such as is done in bio-microarray applications.

According to certain embodiments, the method of the invention may further comprise a step of negative selection of peptides binding to the solid support and/or the shielding peptide. In certain affinity selection procedures the use of such a negative selection step may result in CD70-binding peptides having improved specificity for CD70. The improvement being relative to CD70-binding peptides obtained in methods not including the negative selection step.

In the negative selection step binder peptides are discarded if they have a higher affinity for the shielding peptide or the solid support than for the target peptide. The negative selection step may be performed prior to or after the capturing step using the target peptide in interaction with the shielding peptide (the primary capturing step). According to certain embodiments the negative selection step is performed prior to the primary capturing step by including a negative capturing step involving binding of the binder peptides of the library to the shielding peptide (immobilized on the solid support) in the absence of the target peptide. In this negative pre-selection step unbound binder peptides are selected for use in the primary capturing step. According to certain other embodiments the negative selection step is performed after (post) the primary capturing step by including a negative capturing step involving binding, of the binder peptides selected in the primary capturing step, to the shielding peptide (immobilized on the solid support) in the absence of the target peptide. In this negative post-selection step unbound binder peptides are selected as the CD70-binding peptides. For performing a negative selection step it is preferred that the immobilization of the target peptide is dependent on its interaction with the shielding peptide (the shielding peptide has a stronger interaction with the solid support than with the target peptide). In this embodiment target peptide may be brought in interaction with the shielding peptide immobilized on the solid support after the pre-selection test or the interaction of the target peptide and the immobilized shielding peptide may be disturbed for the post-selection step.

In the procedure of the method of the invention described above, CD70-binding peptides are identified and/or isolated. In order to facilitate production of the CD70-binding peptides it may be beneficial to determine and/or isolate a peptide sequence of a selected CD70-binding peptide and/or a nucleotide sequence coding for the CD70-binding peptides identified and/or obtained with the method. This enables transfection of the nucleotide sequence coding for the CD70-binding peptides to obtain organisms capable of producing the CD70-binding peptides with good efficiency. Depending on the library of binder peptides used, the nucleotide sequence coding for the CD70-binding peptides may be determined and/or isolated with various methods available to the skilled person.

In case the library is a collection of lymphocytes collected from an immunized mammal the CD70-binding peptide will be an immunoglobulin molecule presented on the cell-surface of a lymphocyte clone obtained. The nucleotide sequence coding for the CD70-binding peptides may be obtained by isolating RNA from a culture of the lymphocyte clone, selectively amplifying the immunoglobulin sequence using immunoglobulin-specific primers followed by sequencing of the selectively amplified sequence.

In case the library is a collection of phages, the selected binding peptide will be an antibody or antibody fragment presented on the surface of the phage. The nucleotide encoding for the CD70-binding peptide may be isolated by isolating DNA from the isolated phages followed by sequencing of the DNA.

In case the library is a collection of mRNAs displayed on a ribosome, the selected binding peptide will be displayed on a ribosome. The nucleotide encoding for the CD70-binding peptide may be isolated by isolating the mRNA bound to the ribosome. The identity of the binding-peptide is determined by direct RNA sequencing or generation of cDNA complementary to the mRNA, followed by sequencing of the selectively amplified sequence.

In case the library is a collection of binding-peptides bound to beads (one-bead-one-compound library), one binding peptide is bound to one bead. The identity of the CD70-binding peptide is determined by recovering the peptides from beads selected in the affinity selection procedure, followed by mass-spectrometry procedures.

It will be clear that in the method of the invention for obtaining CD70-binding peptides, reactions and processes such as the binding affinity selection process and associated processes such as capturing steps and washing steps may be performed in a suitable container, such as a reaction vessel.

The invention further relates to a CD70-binding peptide obtainable with the method according to the invention for obtaining CD70-binding peptides. It will be clear to the skilled person that with the method of the invention a great number of different CD70-binding peptides may be obtained. The binding peptides obtainable with the method of the invention share the common feature that, compared to known CD70-binding peptides, they have a reduced inhibition of the CD27-CD70 interaction. CD70-binding peptides, such as antibodies, of the present invention will usually have an EC50 for their target of about below 10⁻³ M, more usually below 10⁻⁶ M, typically below 10⁻⁷ M, more typically below 10⁻⁸ M, preferably below 10⁻⁹ M, and more preferably below 10⁻¹⁰ M, and most preferably below 10⁻¹¹ M. See, e.g. Presta, et al., 2001, Thromb. Haemost. 85:379-389; Yang, et al., 2001, Crit. Rev. Oncol. Hematol. 38:17-23; Carnahan, et al., 2003, Clin. Cancer Res. (Suppl.) 9:3982s-3990s. According to certain embodiments the EC50 of the CD70-binding peptides, such as antibodies, of the invention for their target (CD70) may be selected from 1·10⁻⁶ to 0.5·10⁻¹¹ M, 1·10⁻⁷ to 0.5·10⁻¹¹ M, 1·10⁻⁸ to 0.5·10⁻¹¹ M, 1·10⁻⁸ to 1·10⁻¹¹ M, preferably 5·10⁻⁹ to 1·10⁻¹¹ M, more preferably 5·10⁻⁹ to 1·10⁻¹⁰ M. Binding affinities of the CD70-binding peptides for their target (CD70) may be determined using standard analysis known to the skilled person, in particular the methods disclosed in example 3. According to certain embodiments the obtainable CD70-binding peptides have an IC50 for inhibition of the CD27-CD70 interaction of at least 5·10⁻⁹ M, such as at least 1·10⁻⁸ M, or at least 5·10⁻⁸ M, such as above 1·10⁻⁷ M and more preferably above 2·10⁻⁷ M and most preferably above 3·10⁻⁷ M. Suitably the IC50 may be within 5·10⁻⁹ to 1·10⁻⁴ M, preferably 8·10⁻⁹ to 1·10⁻⁴ M, such as 1·10⁻⁸ to 1·10⁻⁶ M, 1·10⁻⁸ to 4·10⁻⁷ M, 2·10⁻⁸ to 2·10⁻⁷ M, 2·10⁻⁷ to 1·10⁻⁴ M, 3·10⁻⁷ to 1·10⁻⁴ M. The IC50 value for inhibition of the CD27-CD70 interaction of the CD70-binding peptides of the invention may be determined in accordance with general tests for determining binding inhibition, in particular the methods exemplified in example 3.

According to certain embodiments the obtainable CD70-binding peptide is an immunoglobulin or a binding fragment of an immunoglobulin. In the present description and the appended claims the terms immunoglobulin and antibody are used as synonyms and are thus interchangeable. The term “antibody” refers to any form of antibody that exhibits a desired activity, in particular binding to a target location. By binding to the target location certain desired effects may be promoted. For example a compound or moiety associated, for example by being bound with the antibody may be targeted to the target location. According to certain embodiments, binding of the antibody to the target location may elicit Fc-mediated effector function on cells it is bound to. In the present invention the target is CD70. Binding of the antibody to the CD70 epitope, is associated with a reduced interference with the CD27-CD70 interaction, in comparison to known CD70-binding antibodies. The term “antibody” is thus used in the broadest sense and specifically covers, but is not limited to, monoclonal antibodies (including full length monoclonal antibodies), polyclonal antibodies, and multispecific antibodies (e.g., bispecific antibodies). Within the present invention a peptide derived from a certain antibody may be considered an antibody analogue. The skilled person will understand that for a proper functioning of an antibody analogue within the context of this invention a derived antibody (or antibody analogue) may comprise antigen binding regions of its originating antibody. Antibody analogues in particular may comprise antibody fragments, antibodies having modified effector function, chimeric antibodies and humanized antibodies as defined below.

“Antibody fragment” and “antibody binding fragment” mean antigen-binding fragments and comparable parts of an antibody, typically including at least a portion of the antigen binding or variable regions of the parental antibody. An antibody fragment retains at least some of the binding specificity of the parental antibody. For this an antibody fragment may comprise a number of CDRs, in particular a number of CDRs of a V_{H} region, such as CDR1, CDR2 and CDR3 of a V_{H} region. In addition to the number of CDRs of a V_{H} region, an antibody fragment may also comprise a number of CDRs of a V_{L} region, such as CDR1, CDR2 and CDR3 of a V_{L} region. According to certain embodiments antibody fragments may comprise CDR1, CDR2 and CDR3 of a V_{H} region in conjunction with CDR1, CDR2 and CDR3 of a V_{L} region. Typically, an antibody fragment retains at least 10% of the parental binding activity when that activity is expressed on a molar basis. Preferably, an antibody fragment retains at least 20%, 50%, 70%, 80%, 90%, 95% or 100% or more of the parental antibody's binding affinity for the target. Therefore, as is clear for the skilled person, “antibody fragments” in many applications may substitute antibodies and the term “antibody” should be understood as including “antibody fragments” when such a substitution is suitable. Examples of antibody fragments include, but are not limited to, Fab, Fab′, F(ab′)2, and Fv fragments; diabodies; linear antibodies; single-chain antibody molecules, e.g., sc-Fv, unibodies or duobodies (technology from Genmab); domain antibodies (technology from Domantis); nanobodies (technology from Ablynx); and multispecific antibodies formed from antibody fragments. Engineered antibody variants are reviewed in Holliger and Hudson, 2005, Nat. Biotechnol. 23:1126-1136.

An “Fab fragment” may be comprised of one light chain and the CH1 and variable regions of one heavy chain. The heavy chain of a Fab molecule cannot form a disulfide bond with another heavy chain molecule.

An “Fc” region contains two heavy chain fragments which may comprise the C_{H}1 and C_{H}2 domains of an antibody. The two heavy chain fragments are held together by two or more disulfide bonds and by hydrophobic interactions of the C_{H}3 domains.

An “Fab′ fragment” contains one light chain and a portion of one heavy chain that contains the V_{H} domain and the C_{H}1 domain and also the region between the C_{H}1 and C_{H}2 domains, such that an interchain disulfide bond can be formed between the two heavy chains of two Fab′ fragments to form a F(ab′)₂ molecule.

An “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains.

The “Fv region” may comprise the variable regions from both the heavy and light chains, but lacks the constant regions.

A “single-chain Fv antibody” (or “scFv antibody”) refers to antibody fragments which may comprise the V_{H} and V_{L} domains of an antibody, wherein these domains are present in a single polypeptide chain. Generally, the Fv polypeptide further may comprise a polypeptide linker between the V_{H} and V_{L} domains which enables the scFv to form the desired structure for antigen binding. For a review of scFv, see Pluckthun, 1994, The Pharmacology of Monoclonal Antibodies, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

A “diabody” is a small antibody fragment with two antigen-binding sites. The fragment may comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al., 1993, Proc. Natl. Acad. Sci. USA 90: 6444-6448.

A “domain antibody fragment” is an immunologically functional immunoglobulin fragment containing only the variable region of a heavy chain or the variable region of a light chain. In some instances, two or more V_{H} regions are covalently joined with a peptide linker to create a bivalent domain antibody fragment. The two V_{H} regions of a bivalent domain antibody fragment may target the same or different antigens.

An antibody fragment of the invention may comprise a sufficient portion of the constant region to permit dimerization (or multimerization) of heavy chains that have reduced disulfide linkage capability, for example where at least one of the hinge cysteines normally involved in inter-heavy chain disulfide linkage is altered with known methods available to the skilled person. In another embodiment, an antibody fragment, for example one that may comprise the Fc region, retains at least one of the biological functions normally associated with the Fc region when present in an intact antibody, such as FcRn binding, antibody half-life modulation, ADCC (antibody dependent cellular cytotoxicity) function, and/or complement binding (for example, where the antibody has a glycosylation profile necessary for ADCC function or complement binding).

The term “chimeric” antibody refers to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (See, for example, U.S. Pat. No. 4,816,567 and Morrison et al., 1984, Proc. Natl. Acad. Sci. USA 81:6851-6855).

As used herein, the term “humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also may comprise at least a portion of an immunoglobulin constant region (Fc), typically that of a human immunoglobulin. The humanized forms of rodent antibodies may essentially comprise the same CDR sequences of the parental rodent antibodies, although certain amino acid substitutions may be included to increase affinity, increase stability of the humanized antibody, or for other reasons. However, as CDR loop exchanges do not uniformly result in an antibody with the same binding properties as the antibody of origin, changes in framework residues (FR), residues involved in CDR loop support, might also be introduced in humanized antibodies to preserve antigen binding affinity (Kabat et al., 1991, J. Immunol. 147:1709).

The term “antibody” also includes “fully human” antibodies, i.e., antibodies that may comprise human immunoglobulin protein sequences only. A fully human antibody may contain murine carbohydrate chains if produced in a mouse, in a mouse cell, or in a hybridoma derived from a mouse cell. Similarly, “mouse antibody” or “rat antibody” refer to an antibody that may comprise only mouse or rat immunoglobulin sequences, respectively. A fully human antibody may be generated in a human being, in a transgenic animal having human immunoglobulin germline sequences, by phage display or other molecular biological methods. Also, recombinant immunoglobulins may also be made in transgenic mice. See Mendez et al., 1997, Nature Genetics 15:146-156. See also Abgenix, Medarex, MeMo and Kymab technologies.

The antibodies of the present invention also include antibodies with modified (or blocked) Fc regions to provide altered effector functions. See, e.g. U.S. Pat. No. 5,624,821; WO2003/086310; WO2005/120571; WO2006/0057702; Presta, 2006, Adv. Drug Delivery Rev. 58:640-656; Vincent and Zurini, Biotechnol. 1, 2012, 7:1444-50; Kaneko and Niwa, Biodrugs, 2011, 25: 1-11. Such modification can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, and a longer half-life would result in less frequent dosing, with the concomitant increased convenience and decreased use of material. See Presta, 2005, J. Allergy Clin. Immunol. 116:731 at 734-35.

The antibodies of the present invention also include antibodies with intact Fc regions that provide full effector functions, e.g. antibodies of isotype IgG1, which induce complement-dependent cytotoxicity (CDC) or antibody dependent cellular cytotoxicity (ADCC) in a cell associated with the target for the antibody.

The CD70-binding peptides, such as CD70-binding antibodies, may be conjugated (e.g., covalently linked) to molecules that improve stability of the antibody during storage or increase the half-life of the peptide in vivo. Examples of molecules that increase the half-life are albumin (e.g., human serum albumin) and polyethylene glycol (PEG). Albumin-linked and PEGylated derivatives of antibodies can be prepared using techniques well known in the art. See, e.g. Chapman, 2002, Adv. Drug Deliv. Rev. 54:531-545; Anderson and Tomasi, 1988, J. Immunol. Methods 109:37-42; Suzuki et al., 1984, Biochim. Biophys. Acta 788:248-255; and Brekke and Sandlie, 2003, Nature Rev. 2:52-62.

The term “hypervariable region,” as used herein, refers to the amino acid residues of an antibody which are responsible for antigen-binding. The hypervariable region may comprise amino acid residues from a “complementarity determining region” or “CDR,” defined by sequence alignment, for example residues 24-34 (L1), 50-56 (L2) and 89-97 (L3) in the light chain variable domain and 31-35 (H1), 50-65 (H2) and 95-102 (H3) in the heavy chain variable domain (see Kabat et al., 1991, Sequences of proteins of Immunological Interest, 5th Ed. Public Health Service, National Institutes of Health, Bethesda, Md.) and/or those residues from a “hypervariable loop” (HVL), as defined structurally, for example, residues 26-32 (L1), 50-52 (L2) and 91-96 (L3) in the light chain variable domain and 26-32 (H1), 53-55 (H2) and 96-101 (H3) in the heavy chain variable domain (see Chothia and Leskl, 1987, J. Mol. Biol. 196:901-917). “Framework” or “FR” residues are those variable domain residues other than the hypervariable region residues as herein defined.

According to certain embodiments a CD70-binding peptide obtainable with the method of the invention, may comprise immunoglobulin V_{H} domains, which may comprise CDR1, CDR2 and CDR3 sequences having at least 60%, such as at least 85%, preferably at least 90%, more preferably at least 95% sequence similarity with amino acid sequences respectively selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82. Such a CD70-binding peptide may be an immunoglobulin, an immunoglobulin binding fragment or a different analogue thereof.

Said CD70-binding peptide, may comprise immunoglobulin V_{H} and V_{L} domains, which may comprise V_{H} CDR1, V_{H} CDR2 V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences respectively selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences respectively selected from SEQ ID NO: 3 and 4, or 13 and 14, or 23 and 24, or 33 and 34, or 43 and 44, or 53 and 54, or 63 and 64, or 73 and 74. DNA sequences coding for these various sequences can be determined by the skilled person on the basis of his knowledge of the genetic code. In table 1 above a number of DNA sequences coding for the V_{H} and V_{L} amino acid sequences is listed. The sequences are provided in the sequence listing. Such a CD70-binding peptide may be an immunoglobulin, an immunoglobulin binding fragment or a different analogue thereof.

As the skilled person will understand, “sequence similarity” refers to the extent to which individual nucleotide or peptide sequences are alike. The extent of similarity between two sequences is based on the extent of identity combined with the extent of conservative changes. The percentage of “sequence similarity” is the percentage of amino acids or nucleotides which is either identical or conservatively changed viz. “sequence similarity”=(% sequence identity)+(% conservative changes).

For the purpose of this invention “conservative changes” and “identity” are considered to be species of the broader term “similarity”. Thus whenever the term sequence “similarity” is used it embraces sequence “identity” and “conservative changes”.

The term “sequence identity” is known to the skilled person. In order to determine the degree of sequence identity shared by two amino acid sequences or by two nucleic acid sequences, the sequences are aligned for optimal comparison purposes (e.g., gaps can be introduced in the sequence of a first amino acid or nucleic acid sequence for optimal alignment with a second amino or nucleic acid sequence). Such alignment may be carried out over the full lengths of the sequences being compared. Alternatively, the alignment may be carried out over a shorter comparison length, for example over about 20, about 50, about 100 or more nucleic acids/bases or amino acids.

The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The degree of identity shared between sequences is typically expressed in terms of percentage identity between the two sequences and is a function of the number of identical positions shared by identical residues in the sequences (i.e., % identity=number of identical residues at corresponding positions/total number of positions×100). Preferably, the two sequences being compared are of the same or substantially the same length.

The percentage of “conservative changes” may be determined similar to the percentage of sequence identity. However, in this case changes at a specific location of an amino acid or nucleotide sequence that are likely to preserve the functional properties of the original residue are scored as if no change occurred.

For amino acid sequences the relevant functional properties are the physico-chemical properties of the amino acids. A conservative substitution for an amino acid in a polypeptide of the invention may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without substantially altering the activity of a protein, particularly in regions of the protein that are not directly associated with biological activity (see, e.g., Watson, et al., Molecular Biology of the Gene, The Benjamin/Cummings Pub. Co., p. 224 (4th Edition 1987)). For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr and vice versa so that a free —OH is maintained; and Gln for Asn and vice versa to maintain a free —NH₂.

Exemplary conservative substitutions in the amino acid sequence of the CD70 binding peptides of the invention can be made in accordance with those set forth below as follows:

Exemplary Conservative Amino Acid Substitutions

| Original residue | Conservative substitution |
|---|---|
| Ala (A) | Gly; Ser |
| Arg (R) | Lys, His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

For nucleotide sequences the relevant functional properties are mainly the biological information that a certain nucleotide carries within the open reading frame of the sequence in relation to the transcription and/or translation machinery. It is common knowledge that the genetic code has degeneracy (or redundancy) and that multiple codons may carry the same information in respect of the amino acid for which they code. For example in certain species the amino acid leucine is coded by UUA, UUG, CUU, CUC, CUA, CUG codons (or TTA, TTG, CTT, CTC, CTA, CTG for DNA), and the amino acid serine is specified by UCA, UCG, UCC, UCU, AGU, AGC (or TCA, TCG, TCC, TCT, AGT, AGC for DNA). Nucleotide changes that do not alter the translated information are considered conservative changes.

The skilled person will be aware of the fact that several different computer programs, using different mathematical algorithms, are available to determine the identity between two sequences. For instance, use can be made of a computer program employing the Needleman and Wunsch algorithm (Needleman et al. (1970)). According to an embodiment the computer program is the GAP program in the Accelrys GCG software package (Accelrys Inc., San Diego U.S.A.). Substitution matrices that may be used are for example a BLOSUM 62 matrix or a PAM250 matrix, with a gap weight of 16, 14, 12, 10, 8, 6, or 4 and a length weight of 1, 2, 3, 4, 5, or 6. The skilled person will appreciate that all these different parameters will yield slightly different results but that the overall percentage identity of two sequences is not significantly altered when using different algorithms.

According to an embodiment the percent identity between two nucleotide sequences is determined using the GAP program in the Accelrys GCG software package (Accelrys Inc., San Diego U.S.A.). A NWSgapdna CMP matrix and a gap weight of 40, 50, 60, 70, or 80 and a length weight of 1, 2, 3, 4, 5, or 6 is used.

In another embodiment, the percent identity of two amino acid or nucleotide sequences is determined using the algorithm of E. Meyers and W. Miller (Meyers et al. (1989)) which has been incorporated into the ALIGN program (version 2.0) (available at the ALIGN Query using sequence data of the Genestream server IGH Montpellier France http://vegajgh.mrs.fr/bin align-guess.cgi) using a PAM120 weight residue table, a gap length penalty of 12 and a gap penalty of 4.

For the present invention it is most preferred to use BLAST (Basic Local Alignment Tool) to determine the percentage identity and/or similarity between nucleotide or amino acid sequences.

Queries using the BLASTn, BLASTp, BLASTx, tBLASTn and tBLASTx programs of Altschul et al. (1990) may be posted via the online versions of BLAST accessible via http://www.ncbi.nlm.nih.gov. Alternatively a standalone version of BLAST (e.g., version 2.2.24 (released 23 Aug. 2010)) downloadable also via the NCBI internet site may be used. Preferably BLAST queries are performed with the following parameters. To determine the percentage identity and/or similarity between amino acid sequences: algorithm: blastp; word size: 3; scoring matrix: BLOSUM62; gap costs: Existence: 11, Extension: 1; compositional adjustments: conditional compositional score matrix adjustment; filter: off; mask: off. To determine the percentage identity and/or similarity between nucleotide sequences: algorithm: blastn; word size: 11; max matches in query range: 0; match/mismatch scores: 2, −3; gap costs: Existence: 5, Extension: 2; filter: low complexity regions; mask: mask for lookup table only.

The percentage of “conservative changes” may be determined similar to the percentage of sequence identity with the aid of the indicated algorithms and computer programs. Some computer programs, e.g., BLASTp, present the number/percentage of positives (=similarity) and the number/percentage of identity. The percentage of conservative changes may be derived therefrom by subtracting the percentage of identity from the percentage of positives/similarity (percentage conservative changes=percentage similarity−percentage identity).

On the basis of the sequence information available for the CD70-binding peptide, further manipulations are possible. If the CD70-binding peptide is an antibody, the antibody DNA also may be modified, for example, by substituting the coding sequence for human heavy- and light-chain constant domains in place of the homologous murine sequences (U.S. Pat. No. 4,816,567; Morrison, et al., 1984, Proc. Natl Acad. Sci. USA, 81:6851), or by covalently joining to the immunoglobulin coding sequence all or part of the coding sequence for non-immunoglobulin material (e.g., protein domains). Typically such non-immunoglobulin material is substituted for the constant domains of an antibody, or is substituted for the variable domains of one antigen-combining site of an antibody to create a chimeric bivalent antibody which may comprise one antigen-combining site having specificity for an antigen and another antigen-combining site having specificity for a different antigen.

A camelized antibody is a heavy chain only antibody that is derived from a mouse antibody. Camelization can be performed following the method of Tanha et al., Protein Eng Des Sel., 2006, 19:503-9.

A humanized antibody has one or more amino acid residues from a source that is non-human. The non-human amino acid residues are often referred to as “import” residues, and are typically taken from an “import” variable domain. Humanization can be performed generally following the method of Winter and co-workers (Jones et al., 1986, Nature 321:522-525; Riechmann et al., 1988, Nature, 332:323-327; Verhoeyen et al., 1988, Science 239:1534-1536), by substituting rodent CDRs or CDR sequences for the corresponding sequences of a human antibody. Accordingly, such “humanized” antibodies are antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in non-human, for example, rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., 1987, J. Immunol. 151:2296; Chothia et al., 1987, J. Mol. Biol. 196:901). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285; Presta et al., 1993, J. Immnol. 151:2623).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Humanization of antibodies is a straightforward protein engineering task. Nearly all murine antibodies can be humanized by CDR grafting, resulting in the retention of antigen binding. See, Lo, Benny, K. C., editor, in Antibody Engineering: Methods and Protocols, volume 248, Humana Press, New Jersey, 2004.

Amino acid sequence variants of humanized anti-CD70 antibodies are prepared by introducing appropriate nucleotide changes into the humanized anti-CD70 antibodies' DNAs, or by peptide synthesis. Such variants include, for example, deletions from, and/or insertions into, and/or substitutions of, residues within the amino acid sequences shown for the humanized anti-CD70 antibodies. Any combination of deletion, insertion, and substitution is made to arrive at the final construct, provided that the final construct possesses the desired characteristics. The amino acid changes also may alter post-translational processes of the humanized anti-CD70 antibodies, such as changing the number or position of glycosylation sites.

A useful method for identification of certain residues or regions of the humanized anti-CD70 antibodies polypeptides that are preferred locations for mutagenesis is called “alanine scanning mutagenesis,” as described by Cunningham and Wells, 1989, Science 244: 1081-1085. Here, a residue or group of target residues are identified (e.g., charged residues such as Arg, Asp, His, Lys, and Glu) and replaced by a neutral or negatively charged amino acid (most preferably alanine or polyalanine) to affect the interaction of the amino acids with CD70 antigen. The amino acid residues demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, Ala scanning or random mutagenesis is conducted at the target codon or region and the expressed humanized anti-CD70 antibodies' variants are screened for the desired activity.

Ordinarily, amino acid sequence variants of the humanized anti-CD70 antibodies will have an amino acid sequence having at least 75% amino acid sequence identity with the original mouse antibody amino acid sequences of either the heavy or the light chain more preferably at least 80%, more preferably at least 85%, more preferably at least 90%, and most preferably at least 95%, 98% or 99%. Identity or homology with respect to this sequence is defined herein as the percentage of amino acid residues in the candidate sequence that are identical with the humanized residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology. The percentage of identity between two sequences can be determined with computer application such as SeqMan II (DNAstar Inc, version 5.05). Using this program two sequences can be aligned using the optimal alignment algorithm of Smith and Waterman (1981) (Journal of Molecular Biology 147: 195-197). After alignment of the two sequences the percentage identity can be calculated by dividing the number of identical amino acids between the two sequences by the length of the aligned sequences minus the length of all gaps.

Antibodies having the characteristics identified herein as being desirable in humanized anti-CD70 antibodies can be screened for inhibitory biologic activity in vitro or suitable binding affinity. To screen for antibodies that bind to the epitope on human CD70, a routine cross-blocking assay such as that described in Antibodies, A Laboratory Manual, Cold Spring Harbor Laboratory, Ed Harlow and David Lane (1988), can be performed. Antibodies that bind to the same epitope are likely to cross-block in such assays, but not all cross-blocking antibodies will necessarily bind at precisely the same epitope since cross-blocking may result from steric hindrance of antibody binding by antibodies binding at overlapping epitopes, or even nearby non-overlapping epitopes.

Alternatively, epitope mapping, e.g., as described in Champe et al., 1995, J. Biol. Chem. 270:1388-1394, can be performed to determine whether the antibody binds an epitope of interest. “Alanine scanning mutagenesis,” as described by Cunningham and Wells, 1989, Science 244: 1081-1085, or some other form of point mutagenesis of amino acid residues in human CD70 may also be used to determine the functional epitope for anti-CD70 antibodies of the present invention. Another method to map the epitope of an antibody is to study binding of the antibody to synthetic linear and CLIPS peptides that can be screened using credit-card format mini PEPSCAN cards as described by Slootstra et al. (Slootstra et al., 1996, Mol. Diversity 1: 87-96) and Timmerman et al. (Timmerman et al., 2007, J. Mol. Recognit. 20: 283-299). The binding of antibodies to each peptide is determined in a PEPSCAN-based enzyme-linked immuno assay (ELISA). Additional antibodies binding to the same epitope as an antibody of the present invention may be obtained, for example, by screening of antibodies raised against CD70 for binding to the epitope, or by immunization of an animal with a peptide which may comprise a fragment of human CD70 which may comprise the epitope sequences. Antibodies that bind to the same functional epitope might be expected to exhibit similar biological activities, such as blocking receptor binding, and such activities can be confirmed by functional assays of the antibodies.

Other CD70-binding peptides binding to the same epitope as an antibody of the present invention may be obtained, for example, by preselecting binding peptides using the selection technology of the invention and a library displaying binding peptides. Binding peptides that bind to the same functional epitope might be expected to exhibit similar biological activities, such as blocking receptor binding, and such activities can be confirmed by functional assays of the antibodies.

As used herein, the term “about” refers to a value that is within an acceptable error range for the particular value as determined by one of ordinary skill in the art, which will depend in part on how the value is measured or determined, i.e. the limitations of the measurement system. For example, “about” can mean within 1 or more than 1 standard deviation per the practice in the art. Alternatively, “about” or “comprising essentially of” can mean a range of up to 20%. Furthermore, particularly with respect to biological systems or processes, the terms can mean up to an order of magnitude or up to 5-fold of a value. When particular values are provided in the application and claims, unless otherwise stated, the meaning of “about” or “comprising essentially of” should be assumed to be within an acceptable error range for that particular value.

A humanized antibody can be selected from any class of immunoglobulins, including IgM, IgG, IgD, IgA, and IgE. Preferably, the antibody is an IgG antibody. Any isotype of IgG can be used, including IgG1, IgG2, IgG3, and IgG4. Variants of the IgG isotypes are also contemplated. The humanized antibody may comprise sequences from more than one class or isotype. Optimization of the necessary constant domain sequences to generate the desired biologic activity is readily achieved by screening the antibodies in the biological assays described in the Examples.

Likewise, either class of light chain can be used in the compositions and methods herein. Specifically, kappa, lambda, or variants thereof are useful in the present compositions and methods.

A CD70 binding peptide, such as an antibody, antibody analogue or antibody fragment, of the invention may also be conjugated with cytotoxic payloads such as cytotoxic agents or radionucleotides such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr and ⁵⁶Fe. Such antibody conjugates may be used in immunotherapy to selectively target and kill cells expressing a target (the antigen for that antibody) on their surface. Exemplary cytotoxic agents include ricin, vinca alkaloid, methotrexate, Psuedomonas exotoxin, saporin, diphtheria toxin, cisplatin, doxorubicin, abrin toxin, gelonin, pokeweed antiviral protein, monomethyl auristatin E, monomethyl auristatin F, Mertansine and pyrrolobenzodiazepine.

The antibodies and antibody fragments of the invention may also be conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

Any method known in the art for conjugating the antibody molecules or protein molecules of the invention to the various moieties may be employed, including those methods described by Hunter et al., 1962, Nature 144:945; David et al., 1974, Biochemistry 13:1014; Pain et al., 1981, J. Immunol. Meth. 40:219; and Nygren, J., 1982, Histochem. and Cytochem. 30:407. Methods for conjugating antibodies and proteins are conventional and well known in the art.

According to certain embodiments the CD70-binding peptide obtainable with the method of the invention is a binding peptide obtainable from a combinatorial peptide library. Such a CD70-binding peptide need not be based on an antibody structure and thus may be a non-antibody binding peptide. Examples include CD70-binding peptide derived from one-bead-one-peptide libraries. Other examples include CD70-binding peptides based on engineered protein scaffolds, such as Adnectins, Affibodies, Anticalins and DARPins.

A further aspect of the invention relates to a cell which may comprise a nucleotide sequence coding for a CD70-binding peptide obtainable with the method of the invention for obtaining CD70-binding peptides. As discussed above the nucleotide sequence coding for a CD70-binding peptide can be determined and/or isolated with different procedures, depending on the library of binder peptides used. Thus nucleotide sequences coding for a CD70-binding peptide of the invention may be obtained. Such nucleotide sequences may be used for transfection of a host-cell. The cell thus may be a genetically modified cell. In particular the cell may be genetically modified by which may comprise the nucleotide coding for the CD70-binding peptide as a heterologous nucleotide sequence.

The host cell may be a cloning host or an expression host. When selected as an expression host, the host cell expression system preferably is capable of and more preferably optimized for production of heterologous peptides, such as antibodies or antibody fragments. The host-cell may be from a unicellular organism or from a multicellular organism and may be selected from E. coli cells, simian COS cells, Chinese hamster ovary (CHO) cells, or myeloma cells that do not otherwise produce a CD70-binding peptide, such as a CD70-binding immunoglobulin protein or a related protein. For transfection, isolated DNA may be inserted into expression vectors, which are then transfected into host cells.

Alternatively, it is also possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA 90:2551; Jakobovits et al., 1993, Nature 362:255-258; Bruggermann et al., 1993, Year in Immunology 7:33; and Duchosal et al., 1992, Nature 355:258.

With the use of the cell according to the invention the CD70-binding peptide may be produced. Thus a further aspect of the invention relates to a process for producing a CD70-binding peptide which may comprise providing cells according to the invention, culturing said cells and allowing the cells to express and preferably secrete the CD70-binding peptide.

The CD70 binding peptide may be isolated from the host cell expression system and various procedures for this are readily available to the skilled person. The specific procedure best suited will depend on the host cell expression system used and the skilled person will be able to make suitable selections on the basis of the common general knowledge available.

When using recombinant techniques, the CD70-binding peptide, for example an antibody (or fragment) can be produced intracellularly, in the periplasmic space, or directly secreted into the medium. If the CD70-binding peptide is produced intracellularly, as a first step, the particulate debris, either host cells or lysed fragments, is removed, for example, by centrifugation or ultrafiltration. Carter et al., 1992, Bio/Technology 10:163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the CD70-binding peptide is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The CD70-binding peptide composition prepared from the cells can be purified using, for example, hydroxylapatite chromatography, gel electrophoresis, dialysis, and affinity chromatography, with affinity chromatography being a particularly advantageous purification technique. The suitability of protein A as an affinity ligand for immoglobulins depends on the species and isotype of any immunoglobulin Fc region that is present in its protein sequence. Protein A can be used to purify antibodies that are based on human Ig.gamma1, Ig.gamma2, or Ig.gamma4 heavy chains (Lindmark et al., 1983, J. Immunol. Meth. 62:1-13). Protein G is recommended for all mouse isotypes and for human .gamma.3 (Guss et al., 1986, EMBO J 5:1567-1575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the CD70-binding peptide is an antibody and may comprise a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

The CD70-binding peptide, for example an immunoglobulin, including a binding fragment of an immunoglobulin, obtainable with the process for production of a CD70-binding peptide is a further aspect of the invention. This CD70-binding peptide in general will have a peptide sequence within the definition of the CD70-binding peptide obtainable with the method for obtaining a CD70-binding peptide. However, differences may be present in respect of post-translation modifications such as glycosylation profiles. For example, antibodies lacking the core fucose residues have been shown to display enhanced ADCC activity. Modulation of glycosylation of patterns of antibodies is known to a skilled person. For example, the GlycoFi technology allows specific modulation of glycosylation of antibodies to display the desired level of Fc-effector function (Beck et al., Expert Opin Drug Discov., 2010, 5:95-111.)

The CD70-binding peptide, obtainable with the process for production of a CD70-binding peptide may be an isolated antibody. An “isolated” peptide is one that has been identified and separated and/or recovered from a component of the environment from which it is obtained. Contaminant components of its originating environment are materials that would interfere with diagnostic or therapeutic uses for the peptide, and may include enzymes, hormones, and other proteinaceous or non-proteinaceous solutes. In some embodiments, the peptide will be purified (1) to represent at least 50%, such as at least 60%, preferably at least 80%, such as, at least 90% purity by weight of protein in the composition containing the peptide, for example as determined by the Lowry method, and most preferably at least 95%, such as at least 99% by weight of protein in the composition containing the peptide, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The term “monoclonal antibody” as used herein refers to an antibody obtained from a population of substantially homogeneous antibodies, i.e., the individual antibodies which may comprise the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example. The monoclonal antibodies herein specifically include “chimeric” antibodies.

Monoclonal antibodies can be made according to knowledge and skill in the art of injecting test subjects with human CD70 antigen and then generating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA.

The CD70-binding peptide obtainable with the process of the invention for producing a CD70-binding peptide may comprise immunoglobulin V_{H} domains, which may comprise CDR1, CDR2 and CDR3 sequences having at least 60%, such as at least 85%, preferably at least 90%, more preferably at least 95% sequence similarity with amino acid sequences respectively selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82. Such a CD70-binding peptide may be an immunoglobulin, an immunoglobulin binding fragment or a different analogue thereof.

Said CD70-binding peptide may comprise immunoglobulin V_{H} and V_{L} domains, which may comprise V_{H} CDR1, V_{H} CDR2 V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences respectively selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences respectively selected from SEQ ID NO: 3 and 4, or 13 and 14, or 23 and 24, or 33 and 34, or 43 and 44, or 53 and 54, or 63 and 64, or 73 and 74. DNA sequences coding for these various sequences can be determined by the skilled person on the basis of his knowledge of the genetic code. In table 1 above a number of DNA sequences coding for the V_{H} and V_{L} amino acid sequences is listed. The sequences are provided in the sequence listing.

A further aspect of the invention relates to a CD70-binding peptide obtainable with the method for obtaining a number of CD70-binding peptides or the process for producing CD70-binding peptide for use as a medicament. The medicament preferably is a medicament for the treatment of cancer, more preferably a medicament for the treatment of a CD70 positive cancer, most preferably a CD70 over-expressing cancer. In view of the reported efficacy in different (mouse) model systems of targeting CD70 as a tumor antigen the CD70-binding peptides according to the invention have promise for use in medicine, in particular for cancer treatment. In such treatments the CD70-binding peptides of the invention have benefits in view of the reduced inhibition of the CD27-CD70 interaction, thereby maintaining the anti-tumor immunity potential encased in this pathway.

Cancers treatable with the CD70 binding peptide of the present invention may for example be selected from leukemia, acute lymphocytic leukemia, acute myelocytic leukemia, myeloblasts promyelocyte, myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system.

For therapeutic applications the CD70-binding peptides may be used as such or as a treatment conjugate. As used herein, a treatment “conjugate” refers to CD70-binding peptide, such as an antibody, or a fragment thereof, conjugated to a therapeutic moiety, such as a bacterial toxin, a cytotoxic drug or a radiotoxin. Toxic moieties can be conjugated to CD70-binding peptides, such as antibodies, of the invention using methods available in the art.

A composition which may comprise a CD70-binding peptide is the subject of a further aspect of the invention. The composition may comprise the CD70-binding peptide together with a carrier. The composition according to certain embodiments preferably is a pharmaceutical composition.

To prepare pharmaceutical or sterile compositions, the CD70-binding peptide, in particular an antibody or fragment thereof, is admixed with a pharmaceutically acceptable carrier and/or excipient, see, e.g., Remington's Pharmaceutical Sciences and U.S. Pharmacopeia: National Formulary, Mack Publishing Company, Easton, Pa. (1984). Formulations of therapeutic and diagnostic agents may be prepared by mixing with physiologically acceptable carriers, excipients, or stabilizers in the form of, e.g., lyophilized powders, slurries, aqueous solutions or suspensions (see, e.g., Hardman, et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: Parenteral Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Toxicity and therapeutic efficacy of the binding compound, in particular antibody, compositions, administered alone or in combination with another agent, such as the usual anti-cancer drugs, can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

Suitable routes of administration include parenteral administration, such as intramuscular, intravenous, or subcutaneous administration and oral administration. Administration of CD70-binding peptides such as antibodies, used in the pharmaceutical composition or to practice the method of the present invention can be carried out in a variety of conventional ways, such as oral ingestion, inhalation, topical application or cutaneous, subcutaneous, intraperitoneal, parenteral, intraarterial or intravenous injection. In one embodiment, the binding compound of the invention is administered intravenously. In another embodiment, the binding compound of the invention is administered subcutaneously.

Alternatively, one may administer the CD70-binding peptide in a local rather than systemic manner, for example, via injection of the CD70-binding peptide directly into the site of action, often in a depot or sustained release formulation. Furthermore, one may administer the antibody in a targeted drug delivery system.

Guidance in selecting appropriate doses of antibodies, cytokines, and small molecules is available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert, et al., 2003, New Engl. J. Med. 348:601-608; Milgrom, et al., 1999, New Engl. J. Med. 341:1966-1973; Slamon, et al., 2001, New Engl. J. Med. 344:783-792; Beniaminovitz, et al., 2000, New Engl. J. Med. 342:613-619; Ghosh, et al., 2003, New Engl. J. Med. 348:24-32; Lipsky, et al., 2000, New Engl. J. Med. 343:1594-1602).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

A preferred dose protocol is one involving the maximal dose or dose frequency that avoids significant undesirable side effects. A total weekly dose is generally at least 0.05 μg/kg body weight, more generally at least 0.2 μg/kg, most generally at least 0.5 μg/kg, typically at least 1 μg/kg, more typically at least 10 μg/kg, most typically at least 100 μg/kg, preferably at least 0.2 mg/kg, more preferably at least 1.0 mg/kg, most preferably at least 2.0 mg/kg, optimally at least 10 mg/kg, more optimally at least 25 mg/kg, and most optimally at least 50 mg/kg (see, e.g., Yang, et al., 2003, New Engl. J. Med. 349:427-434; Herold, et al., 2002, New Engl. J. Med. 346:1692-1698; Liu, et al., 1999, J. Neurol. Neurosurg. Psych. 67:451-456; Portielji, et al., 2003, Cancer Immunol. Immunother. 52:133-144). The desired dose of a small molecule therapeutic, e.g., a peptide mimetic, natural product, or organic chemical, is about the same as for an antibody or polypeptide, on a moles/kg basis.

“Administration”, “therapy” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration”, “therapy” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration”, “therapy” and “treatment” also mean in vitro and ex vivo treatments, e.g., of a cell, by a reagent, diagnostic, binding composition, or by another cell.

As used herein, “inhibit” or “treat” or “treatment” includes a postponement of development of the symptoms associated with disease and/or a reduction in the severity of such symptoms that will or are expected to develop with said disease. The terms further include ameliorating existing symptoms, preventing additional symptoms, and ameliorating or preventing the underlying causes of such symptoms. Thus, the terms denote that a beneficial result has been conferred on a vertebrate subject with a disease.

As used herein, the term “therapeutically effective amount” or “effective amount” refers to an amount of a CD70-binding peptide, such as an anti-CD70 antibody or fragment thereof, that when administered alone or in combination with an additional therapeutic agent to a cell, tissue, or subject is effective to prevent or ameliorate the disease or condition to be treated. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. An effective amount of therapeutic will decrease the symptoms typically by at least 10%; usually by at least 20%; preferably at least about 30%; more preferably at least 40%, and most preferably by at least 50%.

Methods for co-administration or treatment with a second therapeutic agent are well known in the art, see, e.g., Hardman, et al. (eds.), 2001, Goodman and Gilman's The Pharmacological Basis of Therapeutics, 10th ed., McGraw-Hill, New York, N.Y.; Poole and Peterson (eds.), 2001, Pharmacotherapeutics for Advanced Practice: A Practical Approach, Lippincott, Williams & Wilkins, Phila., Pa.; Chabner and Longo (eds.), 2001, Cancer Chemotherapy and Biotherapy, Lippincott, Williams & Wilkins, Phila., Pa.

The pharmaceutical composition of the invention may also contain other agents, including but not limited to a cytotoxic, chemotherapeutic, cytostatic, anti-angiogenic or antimetabolite agents, a tumor targeted agent, an immune stimulating or immune modulating agent or an antibody conjugated to a cytotoxic, cytostatic, or otherwise toxic agent. The pharmaceutical composition can also be employed with other therapeutic modalities such as surgery, chemotherapy and radiation.

Apart from use as a treatment agent, the CD70-binding peptides according to the invention may also find use as a diagnostic tool and/or an analytical tool. Thus further aspects of the invention relate to such uses of the CD70-binding peptide. For example the CD70-binding peptide may be used for detecting expression of CD70 on specific cells, tissues, or in serum. For diagnostic applications, the CD70-binding peptide of the invention typically will be linked (either directly or indirectly) to a detectable labeling group, the signaling moiety. Numerous labeling moieties are available which can be generally grouped into the following categories: biotin, fluorochromes, radionucleotides, enzymes, iodine, and biosynthetic labels. The potential of the CD70-binding peptides of the present invention for use in diagnostic and analytical applications is further supported by the results presented in the experimental section.

The CD70-binding peptides of the present invention may be employed in any known assay method, such as competitive binding assays, direct and indirect sandwich assays, and immunoprecipitation assays (Zola, Monoclonal Antibodies. A Manual of Techniques, pp. 147-158 (CRC Press, Inc. 1987)).

The CD70-binding peptides of the invention may also be used for in vivo diagnostic assays. Generally, the CD70-binding peptide is labeled with a radionuclide so that a CD70 antigen or cells expressing it can be localized using immunoscintigraphy or positron emission tomography.

The CD70-binding peptides of the invention may also have other, non-therapeutic uses. The non-therapeutic uses for the CD70-binding peptides include flow cytometry, western blotting, enzyme linked immunosorbant assay (ELISA) and immunohistochemistry.

CD70-binding peptides of this invention may for example also be used as an affinity purification reagent via immobilization to a Protein A-Sepharose column.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Commercially Available Anti-hCD70 Antibodies Block the CD27 Interaction

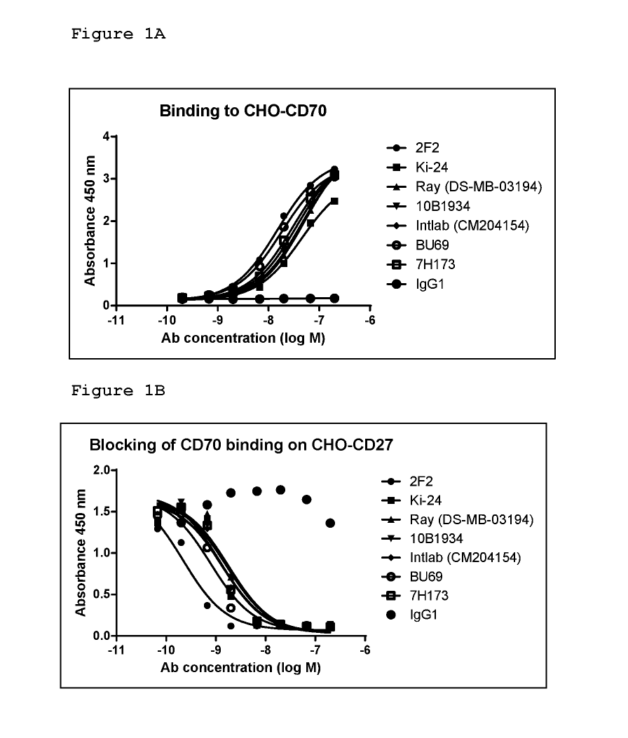

To confirm that reported anti-hCD70 antibodies block the CD27-CD70 interaction, blocking properties were established using cell-based ELISA experiments. First, cell-based ELISA experiments using the commercially available anti-hCD70 antibodies (see Table 2) were performed to determine binding activities of these anti-hCD70 antibodies to cellularly expressed hCD70. In this cell-ELISA, all incubation steps were followed by a wash step with PBST (PBS with 0.01% Tween 20). CHO-K1.hCD70 cells were seeded (40,000 cells/well) in tissue culture plates and incubated overnight at 37° C. The next day, culture medium was removed and cells were incubated for one hour with (dilutions of) purified antibodies at 37° C. Next, cells were washed three times with PBST and incubated for one hour at 37° C. with 1:1,000 goat-anti-mouse IgG-HRP (Southern Biotechnology, #1030-05). Subsequently, cells were washed 6 times with PBST and anti-hCD70 immunoreactivity was visualized with 100 μl TMB Stabilized Chromagen (Invitrogen, cat. no. SB02). Reactions were stopped with 100 μl 0.5 M H₂SO₄ and absorbances were read at 450 and 620 nm. As shown in FIG. 1A, the different anti-hCD70 antibodies bound to hCD70 with similar binding strengths. Calculated EC₅₀, representing the concentration at which 50% of the total binding signal is observed are represented in Table 2.

Blocking properties of the purified antibodies were studied using a cell-based competition assay. This assay works along the following principles: CHO-K1.CD27 cells were seeded (40,000 cells/well) in a 96-well plate and incubated overnight at 37° C. After medium removal, 50 μl recombinant hCD70 (CD70(h)-muCD8 fusion Protein (Ancell, cat. no. ANC-537-030) (0.5 μg/ml) and 50 μl of different dilutions of purified anti-hCD70 antibodies were added. After 1 hour incubation at room temperature, the wells were washed 3 times with PBST. Next, 100 μl/well Streptavidin-HRP conjugate (BD Pharmingen, cat. no. 554066) (1:1,000) was added and cells were incubated for one hour at 37° C. After 6 final washes with PBST TMB Stabilized Chromagen (Invitrogen, cat. no. SB02) (100 μl/well) was added. The reaction was stopped by the addition 100 μl 0.5 M H₂SO₄. Absorbencies were read at 450 and 620 nm. As shown in FIG. 1B, all commercially available anti-hCD70 antibodies blocked the interaction between recombinant human CD70 and CHO-K1.CD27 cells. Calculated IC₅₀, representing the concentration at which 50% of the total binding signal is inhibited is observed are represented in Table 2.

TABLE 2
Commercially available anti-CD70 antibodies

| Antibody | Company | Cat no. | EC50 (nM) | IC50 (nM) |
|---|---|---|---|---|
| 2F2 | Pelicluster | M1705 | 0.014 | 0.4 |
| (CLB70/2) | | | | |
| Ki-24 | BD | 555833 | 0.035 | 1.5 |
| DS-MB-03194 | Ray Biotech | DS-MB-03194 | 0.045 | 1.8 |
| 10B1934 | US | C2425-02 | 0.04 | 1.7 |
| | Biologicals | | | |
| CM204154 | Int. Lab | CM204154 | 0.033 | 1.5 |
| BU69 | Santa Cruz | Sc-65271 | 0.017 | 0.96 |
| 7H173 | LifeSpan | LS-C35733 | 0.026 | 1.4 |
| | Biosciences | | | |

### Example 2

### Immunization and Selection of Anti-hCD70 Antibodies

Immunization of Mice with hCD70 cDNA

To isolate antibodies against the human CD70 protein that harbor reduced-blocking activity towards CD27 binding mice were immunized with hCD70 cDNA. Next, selection procedures were designed and developed to specifically isolate B-cells expressing anti-hCD70 with reduced-blocking activity.

Anti-hCD70 antibodies were raised by cDNA immunization of mice. First, the cDNA encoding the full length open reading frame of hCD70 was subcloned into the pCI-neo vector (Promega, Madison, Wis.). Expression of the obtained vector was checked by transient transfection of pCI-neo-hCD70 in CHO-K1 cells (American Type Culture Collection, Manassas, Va.) and flow cytometry using 10 μg/ml mouse anti-hCD70 IgG1 (BD Pharmingen #555833), followed by goat anti-mouse IgG-FITC (1:100) (Southern Biotechnology, Birmingham, Ala.). Mice were immunized by gene gun immunization using a Helios Gene gun (BioRad, Hercules, Calif.) and DNA coated gold bullets (BioRad) following manufacturer's instructions. Briefly, 1 μm gold particles were coated with pCI-neo-hCD70 cDNA and commercial expression vectors for mouse Flt3L and mouse GM-CSF in a 2:1:1 ratio (both from Aldevron, Fargo, N. Dak.). A total of 1 μg of plasmid DNA was used to coat 500 μg of gold particles.

Specifically, 7-8 weeks old female BALB/C mice were immunized in the ears with a gene gun, receiving 3 cycles of a shot in both ears. Approximately, a 1:8,000 anti-hCD70 titer was detected by cell-ELISA in mouse serum after two DNA immunizations. In the cell-ELISA, all incubation steps were followed by a wash step with PBST (PBS with 0.01% Tween 20). CHO-K1.hCD70 cells were seeded (40,000 cells/well) in tissue culture plates and incubated overnight at 37° C. The next day, culture medium was removed and cells were incubated for 1 hour with (dilutions of) mouse serum at 37° C. Next, cells were washed three times with PBST and incubated for 1 hour at 37° C. with 1:1,000 goat-anti-mouse IgG-HRP (Southern Biotechnology, #1030-05).

Subsequently, cells were washed 6 times with PBST and anti-hCD70 immunoreactivity was visualized with 100 μl OptiEIA TMB substrate (BD Biosciences, Franklin Lake, N.J.). Reactions were stopped with 100 μl 0.5 M H₂SO₄ and absorbances were read at 460 and 620 nm. Mice that demonstrated reactivity against hCD70 were immunized for a final, fourth time and sacrificed four days later.

Erythrocyte-depleted spleen cell populations were prepared as described previously (Steenbakkers et al., 1992, J. Immunol. Meth. 152: 69-77; Steenbakkers et al., 1994, Mol. Biol. Rep. 19: 125-134) and frozen at −140° C.

### Selection of Reduced-Blocking Anti-hCD70 Antibody Producing B Cells

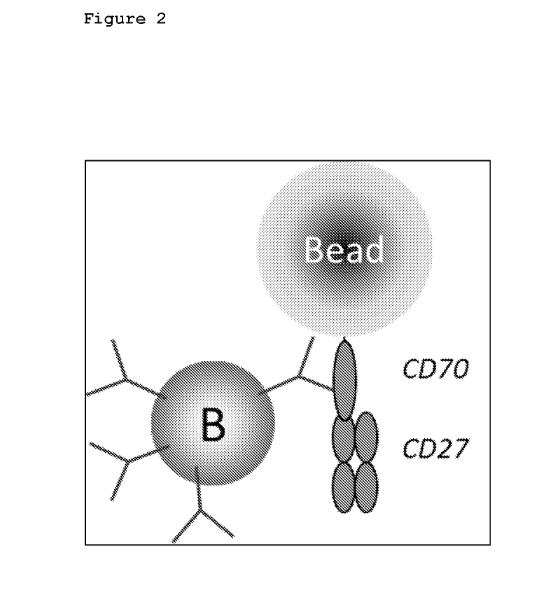

To specifically select the reduced-blocking anti-hCD70 antibody producing B-cells, a selection strategy was designed and developed that preferentially bound B-cells that express reduced-blocking anti-hCD70 antibodies. 5×10⁷ M-280 Streptavidin magnetic Dynabeads (Cat 112.06D) were incubated for 4 hours with 10 μg recombinant hCD70 (CD70(h)-muCD8 fusion Protein (Ancell, cat. no. ANC-537-030) in 500 μl PBS/1% BSA. Next, the supernatant was aspirated and after two washes with PBS/1% BSA, 10 μg hCD27-Fc recombinant protein (R&D systems, 382-CD) in 500 μl PBS/1% BSA was allowed to bind (FIG. 2). After overnight incubation CD70-CD27 complexed beads were 10× washed with 5 ml of DMEM F12/P/S/10% BCS medium. As a negative selection, 5×10⁷ M-280 Streptavidin magnetic Dynabeads (Cat 112.06D) were incubated in 500 μl PBS/1% BSA. Next, the supernatant was aspirated and after two washes with PBS/1% BSA, 10 μg hCD27-Fc recombinant protein (R&D systems, 382-CD) in 500 μl PBS/1% BSA was allowed to bind. After overnight incubation CD70-CD27 complexed beads were 10× washed with 5 ml of DMEM F12/P/S/10% BCS medium.

To select B cell clones producing reduced-blocking anti-hCD70 antibodies, 3.5×10⁷ erythrocyte-depleted splenocytes were thawn. hCD70-specific B-cells were selected by subjecting the splenocytes to negative selection (BSA-blocked streptavidin magnetic Dynabeads), followed by positive selection on CD70-CD27 complexed streptavidin magnetic DynaBeads in a beads: cells ratio of 1.5:1. A specific binding splenocytes were washed away by 10× washes with 5 ml of DMEM F12/P/S/10% BCS medium. In parallel, hCD70-specific B-cells were selected by subjecting the splenocytes only to positive selection on CD70-CD27 complexed streptavidin magnetic DynaBeads in a beads: cells ratio of 1.5:1. A specific binding splenocytes were washed away by 10× washes with 5 ml of DMEM F12/P/S/10% BCS medium. Next, selected B-cells (using both strategies) were cultured as described by Steenbakkers et al., 1994, Mol. Biol. Rep. 19: 125-134. Briefly, selected B-cells were mixed with 7.5% (v/v) T-cell supernatant and 50,000 irradiated (2,500 RAD) EL-4 B5 nursing cells in a final volume of 200 μl DMEM F12/P/S/10% BCS in a 96-well flat-bottom tissue culture plates.

On day eight, supernatants were screened for hCD70 reactivity by cell-ELISA as described above. Twenty B-cell clones expressing hCD70-reactive antibodies were identified by cell-ELISA. All incubation steps were followed by a wash step with PBST (PBS with 0.01% Tween 20). CHO-K1.hCD70 cells were seeded (40,000 cells/well) in tissue culture plates and incubated overnight at 37° C. The next day, culture medium was removed and cells were incubated for one hour with (dilutions of) B-cell supernatant at 37° C. Next, cells were washed three times with PBST and incubated for one hour at 37° C. with 1:1,000 goat-anti-mouse IgG-HRP (Southern Biotechnology, #1030-05). Subsequently, cells were washed 6 times with PBST and anti-hCD27 immunoreactivity was visualized with 100 μl TMB Stabilized Chromagen (Invitrogen, cat. no. SB02). Reactions were stopped with 100 μl 0.5 M H₂SO₄ and absorbances were read at 450 and 620 nm.

Subsequently, the B-cell clones from the hCD70 reactive supernatants were immortalized by mini-electrofusion following published procedures (Steenbakkers et al., 1992, J. Immunol. Meth. 152: 69-77; Steenbakkers et al., 1994, Mol. Biol. Rep. 19:125-34). Specifically, B-cells were mixed with 10⁶ Sp2/0-Ag14 myeloma cells, and serum was removed by washing with DMEM F12 media. Cells were treated with Pronase solution (Calbiochem, cat. no. 4308070.536) for 3 minutes and washed with Electrofusion Isomolar Buffer (Eppendorf, cat. no. 53702). Electrofusions were performed in a 50 μl fusion chamber by an alternating electric field of 30 s, 2 MHz, 400 V/cm followed by a square, high field pulse of 10 μs, 3 kV/cm and again by an alternating electric field of 30 s, 2 MHz, 400 V/cm.

Contents of the chamber were transferred to hybridoma selective medium and plated in a 96-well plate under limiting dilution conditions. On day 12 following the fusions, hybridoma supernatants were screened for hCD70-binding activity, as described above. Nine hybridomas that secreted antibodies in the supernatant that recognized hCD70 were subcloned by limited dilution to safeguard their integrity. The following anti-hCD70 antibodies were selected for further analysis: hCD70.17, hCD70.21, hCD70.23, hCD70.27, hCD70.29, hCD70.32, hCD70.34, hCD70.36 and hCD70.39.

The selection strategy used to identify the CD70-binding peptides is schematically presented in FIG. 2. In this schematic figure the target peptide (CD70) is bound (or otherwise immobilized) to the solid support (Bead) and the shielding peptide (CD27) is immobilized on the solid support by its interaction with the target peptide. However, as is clear from the description above, in alternative embodiments the shielding peptide may be bound (or otherwise immobilized) to the solid support and the target peptide may be immobilized on the solid support by the interaction with the shielding peptide.

### Example 3

### Purification and Characterization of Anti-hCD70 Antibodies

### Stabilization of Anti-hCD70 Producing Hybridomas and Purification of Anti-hCD70 Antibodies

Clonal cell populations were obtained for the hCD70 hybridomas by two rounds of limiting dilutions. Stable hybridomas were cultured in serum-free media for 7-10 days; supernatants were harvested and filtered through a 0.22 μM nitrocellulose membrane. Antibodies were purified using Prosep A spin columns according to the manufacturer's instructions (Millipore, cat. no. LSK2ABA60). Buffer was exchanged for PBS using PD-10 gel-filtration columns (GE Healthcare). Antibodies were concentrated with Amicon Ultra-15 centrifugal filter units (Millipore, Billerica, Mass.) and quantified using spectrophotometry. Using a mouse monoclonal antibody isotyping test kit (Roche, #11493027001), the (sub)-isotype of all hCD70 antibodies was determined to be IgG1, Kappa.

### Binding Analysis

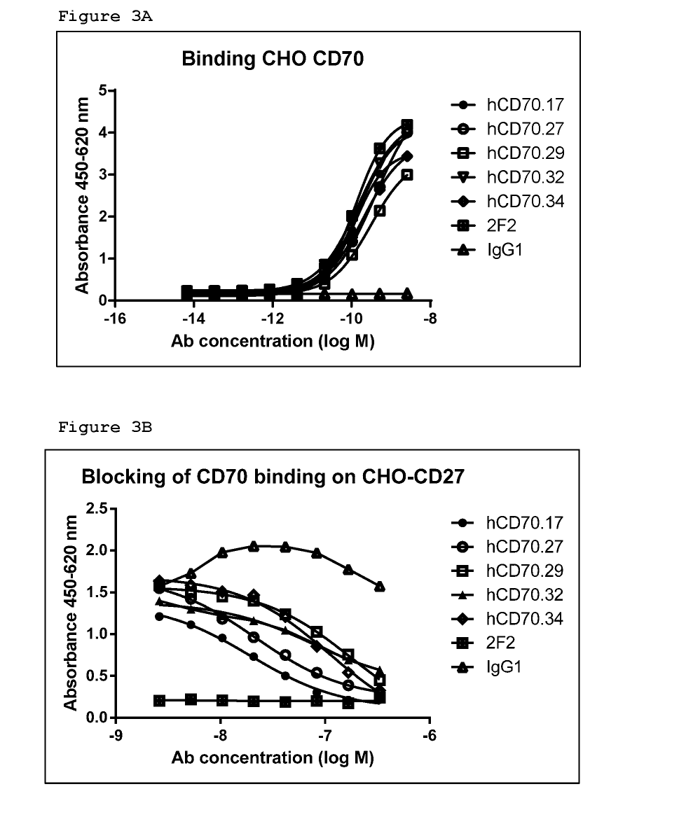

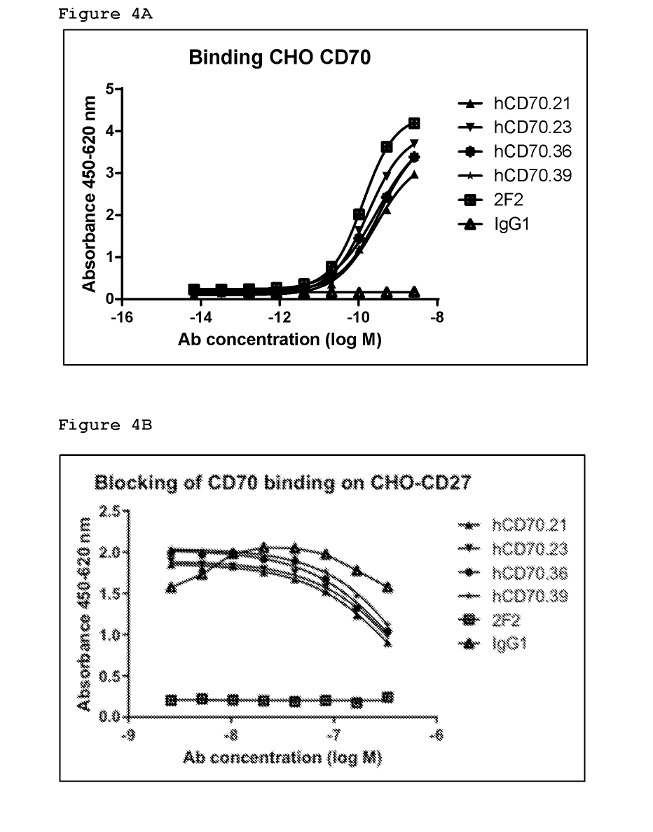

Cell-based ELISA experiments using purified hCD70 antibodies were performed to determine binding activities of hCD70 to cellularly expressed hCD70. In this cell-ELISA, all incubation steps were followed by a wash step with PBST (PBS with 0.01% Tween 20). CHO-K1.hCD70 cells were seeded (40,000 cells/well) in tissue culture plates and incubated overnight at 37° C. The next day, culture medium was removed and cells were incubated for one hour with (dilutions of) purified antibodies at 37° C. Next, cells were washed with PBST and incubated for one hour at 37° C. with 1:1,000 goat-anti-mouse IgG-HRP (Southern Biotechnology, #1030-05). Subsequently, cells were washed 6 times with PBST and anti-hCD70 immunoreactivity was visualized with 100 μl TMB Stabilized Chromagen (Invitrogen, cat. no. SB02). Reactions were stopped with 100 μl 0.5 M H₂SO₄ and absorbances were read at 450 and 620 nm. As shown in FIGS. 3A and 4A, the different hCD70 antibodies bound to hCD70 with similar binding strengths. Calculated EC₅₀, representing the concentration at which 50% of the total binding signal is observed are represented in Table 3 using 2F2 anti-hCD70 antibody (Pelicluster) as a reference.

TABLE 3

| | | IC₅₀ (nM) | Binding |
|---|---|---|---|
| | | CHO-K1.hCD27 | Wil-2S |
| | EC₅₀ (nM) | vs. | Daudi |
| | CHO-K1.hCD70 | CD8-hCD70 | RAJI |
| hCD70.21 | 0.26 | >330 | +/− |
| hCD70.23 | 0.17 | >330 | ++ |
| hCD70.39 | 0.34 | >330 | + |
| hCD70.36 | 0.30 | >330 | + |
| hCD70.32 | 0.15 | 76 | ++ |
| hCD70.29 | 0.31 | 179 | + |
| hCD70.34 | 0.21 | 109 | ++ |
| hCD70.17 | 0.12 | 19 | ++ |
| hCD70.27 | 0.41 | 21 | + |

Reference antibodies

| | | | |
|---|---|---|---|
| 2F2 | 0.13 | <2.6 | Not |
| | | | determined |
| Ki-24 | | | ++ |

Blocking properties of the purified antibodies were studied using a cell-based competition assay. The CHO-K1.CD27 assay works along the following principles: CHO-K1.CD27 cells were seeded (40,000 cells/well) in a 96-well plate and incubated overnight at 37° C. After medium removal, 50 μl recombinant hCD70 (CD70(h)-muCD8 fusion Protein (Ancell, cat. no. ANC-537-030) (0.5 μg/ml) and 50 μl of different dilutions of purified anti-hCD70 antibodies were added.

After 1 hour incubation at room temperature, the wells were washed 3 times with PBST. Next, 100 μl/well Streptavidin-HRP conjugate (BD Pharmingen, cat. no. 554066) (1:5,000) was added and cells were incubated for one hour at 37° C. After 6 final washes with PBST TMB Stabilized Chromagen (Invitrogen, cat. no. SB02) (100 μl/well) was added. The reaction was stopped by the addition of 100 μl 0.5 M H₂SO₄. Absorbencies were read at 460 and 620 nm. Reference antibody: anti-hCD70, clone 2F2 (Pelicluser). As shown in FIGS. 3B and 4B, the purified hCD70 antibodies reduced the blocking of the CD27-CD70 interaction. Calculated IC₅₀ values of hCD70 antibodies and the reference 2F2 anti-hCD70 antibody, which represent the concentration at which half of the inhibition is observed, are presented in Table 3.

### Example 4

### Reduced-Blocking Anti-hCD70 Antibodies Demonstrate Tumor Killing of CD70+ Tumor Cells

### Reduced-Blocking Anti-hCD70 Antibodies Bind to CD70+ Tumor Cells

To study the binding of the isolated reduced-blocking anti-hCD70 antibodies to CD70+ tumor cells, WIL2-S, Daudi and Raji cell-lines were obtained from American Type Culture Collection (Manassas, Va.) and cultured in RPMI 1640 (Gibco, Ref#52400), Pen/Strep (Gibco, Ref#15140-122), 10% Foetal Bovine Serum (Hyclone, Lot# DRE0250), 1% Sodium Pyruvate (Gibco, Ref#11360, only for WIL2-S). For binding analyses dilutions of hCD70 antibodies were made in PBS/1% BSA. 100-200,000 cells/well seeded in a round bottom plate and pelleted by centrifugation. Supernatants were removed by flicking the plate and 100 μl of diluted antibodies were added and incubated for 1 hour at 4° C. Next, cells were 2× washed with PBS/1% BSA and 1 μg of Goat anti-mouse Ig FITC (BD Pharmingen, 349031) was added in 100 μl PBS/1% BSA followed by 30 minute incubation at 4° C. in the dark. Finally, cells were 2× washed with PBS/1% BSA before analysis of bound anti-hCD70 antibodies using flow cytometry (FACScantoII). Mouse IgG, k Isotype control (eBiosciences, 16-4714-85) was used as an isotype control. Acquired data were analyzed using Flowjo v10.0.5. All reduced-blocking anti-hCD70 antibodies bind to B-cell derived tumor cell-lines (Table 3).

### Reduced-Blocking Anti-hCD70 Antibodies Demonstrate Tumor Killing of CD70+ Tumor Cells

To study the ability of the anti-hCD70 antibodies to induce complement-mediated cell death of CD70+ tumor cells, CD70+ tumor cells (e.g. WIL2S, Raji, Daudi) were first loaded with Calcein AM (Invitrogen, C3099) in PBS in a final concentration of around 1 μg/ml by incubation at 37° C. for 30 minutes. Loaded cells are pelleted by centrifugation and resuspended in RPMI 1640 (Gibco, #52400). Next, 30,000 cells are seeded per well in a round-bottom 96-wells plate. Serial dilutions of anti-hCD70 antibodies in RPMI 1640 medium are added. Finally, complement (e.g. human complement (Sigma, S17664-1ML) or Low Tox-M Rabbit complement (Cedarlane, CL3051) is added in a concentration range (for example 16%-50% complement) and incubated for two hours at 37° C. Complement induced cell cytotoxicity is assessed after labeling with propidium iodide (BD Pharmingen, 51-66211E) by flow cytometry. Calcein-positive, Propidium iodide-negative cells represent live cells, while Calcein-negative cells represent dying cells.

To study the ability of the anti-hCD70 antibodies to induce antibody-dependent cell-cytotoxicity of CD70+ tumor cells, CD70+_ tumor cells (e.g. WIL2S, Raji, Daudi) were first loaded with Calcein AM (Invitrogen, C3099) in PBS in a final concentration of around 1 μg/ml by incubation at 37° C. for 30 minutes. Loaded cells are pelleted by centrifugation and resuspended in RPMI 1640 (Gibco, #52400). 200,000 PBMCs are seeded per well in a round-bottom 96-wells plate. Subsequently, serial dilutions of anti-hCD70 antibodies in RPMI 1640 medium supplemented with 10% Foetal Calf serum was added. Finally, calcein-loaded CD70+_ tumor cells (e.g. WIL2S, Raji, Daudi) are added in different effector: target rations (e.g. 100:1 or 50:1 or 25:1). Cells are incubated at 37° C. for 4.5 hours and cell viability was analyzed after labeling with propidium iodide (BD Pharmingen, 51-66211E) by flow cytometry. Calcein-positive, Propidium iodide-negative cells represent live cells, while Calcein-negative cells represent dying cells.

### Example 5

### Reduced-Blocking Anti-hCD70 Antibodies do not Block T-Cell Activation

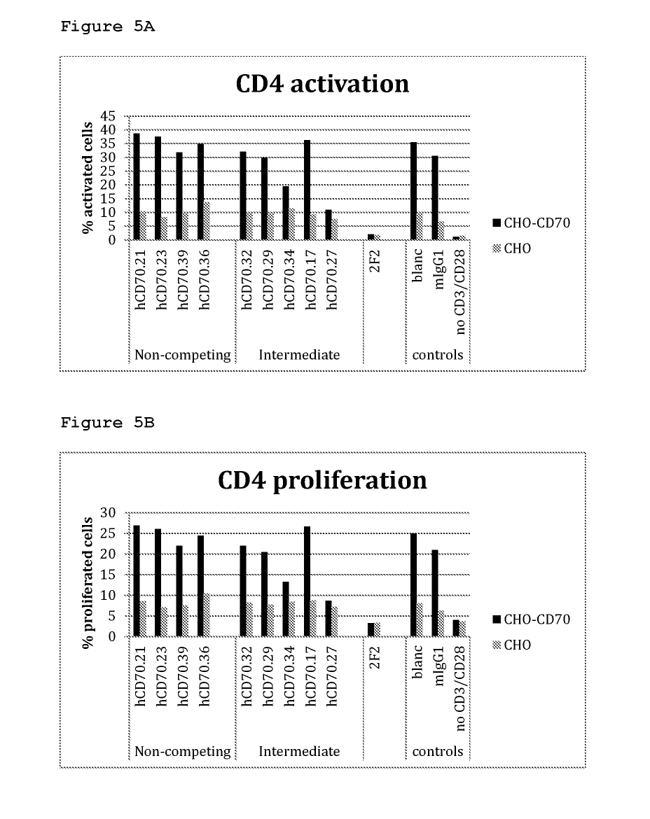

To study the effect of reduced-blocking anti-CD70 antibodies on T-cell activation a co-culture assay of CHO-K1.CD70 cells and human CD4⁺ T-cells was developed. 40,000 irradiated (3,000 RAD) CHO-K1 or CHO-K1.CD70 cells were plated per well in 96-well plates. The next day, isolated CD4+CD25− cells were loaded with 0.5 μM CFSE (Invitrogen, C34554) by incubation on ice for 10 minutes in PBS. Cells were washed with DMEM medium (Gibco, 11320) supplemented with 10% Foetal Calf serum and plated in the wells that had been seeded with CHO-K1 or CHO-K1.CD70 cells. Next, a dilution range of anti-CD70 antibodies was added in medium. Finally, anti-CD3 and anti-CD28 antibodies were added to a final concentration of 0.125 and 1 μg/ml, respectively and co-culture were incubated for 4 days at 37° C., 5% CO₂ and 95% humidity. After 4 days, cells were resuspended and labeled with Propidium Iodide (BD Pharmingen, 556463). Proliferation was assessed by flow cytometry, using CFSE dilution to detect proliferated cells and Propidium Iodide exclusion to detect live cells. As shown in FIG. 5, the purified anti-hCD70 antibodies reduced blocking of the CD70 mediated CD4⁺ T-cell proliferation.

### Example 6

### Characterization of Reduced-Blocking Anti-hCD70 Antibodies

### Epitope Mapping

Synthesis of Peptides and Pepscan Screening

The synthetic linear and CLIPS peptides were synthesized and screened using credit-card format mini PEPSCAN cards (455-well plate with 3 ul wells) as described by Slootstra et al. (Slootstra et al., 1996, Mol. Diversity 1: 87-96) and Timmerman et al. (Timmerman et al., 2007, J. Mol. Recognit. 20: 283-299). The binding of antibodies to each peptide was tested in a PEPSCAN-based enzyme-linked immuno assay (ELISA). The 455-well creditcard-format polypropylene cards, containing the covalently linked peptides, were incubated with sample (for example 1 ug/ml antibody diluted in a PBS solution containing 5% horse serum (vol/vol) and 5% ovalbumin (weight/vol)) and 1% Tween 80 (4° C., overnight). After washing the peptides were incubated with an anti-antibody peroxidase (dilution 1/1000, for example rabbit anti-mouse peroxidase, Southern Biotech) (1 hour, 25° C.), and subsequently, after washing the peroxidase substrate 2,2′-azino-di-3-ethylbenzthiazoline sulfonate (ABTS) and 2, ul/ml 3% H₂O₂ were added. After 1 hour the color development was measured. The color development of the ELISA was quantified with a CCD-camera and an image processing system. The setup consists of a CCD-camera and a 55 mm lens (Sony CCD Video Camara XC-77RR, Nikon micro-nikkor 55 mm f/2.8 lens), a camera adaptor (Sony Camara adaptor DC-77RR) and Image Processing Software.

Synthesis Peptides

The TNF-homology domain was used to develop a molecular model of CD70. Based on this model a total of about 1500, linear and CLIPS peptides were synthesized.

The following CLIPS topologies were used: T2 CLIPS couples to the side-chain of two cysteines to form a single loop topology, while T3 CLIPS couples to the side-chain of three cysteines to form double loop topology, while T2T2 CLIPS first T2 couples to two cysteines (labeled C), and second T2 couples to two cysteines and finally T2T3 CLIPS T2 couples to two cysteines and T3 couples to three cysteines.

Data Analysis and Epitope Determination

Each antibody was tested on all peptides and their binding values were ranked. Clearly re-occurring sequences in most the top binders (˜top 1%) were considered as epitope candidates.

Cloning of Immunoglobulin cDNAs

Degenerate primer PCR-based methods were used to determine the DNA sequences encoding the variable regions for the mouse antibody that is expressed by the hCD70 hybridomas: hCD70.17, hCD70.21, hCD70.23, hCD70.27, hCD70.29, hCD70.32, hCD70.34, hCD70.36 and hCD70.39.

Total RNA was isolated from about 5×10⁶ hybridoma cells using RNeasy mini kit (Qiagen, 74106) according to manufacturer's instructions, and treated with Deoxyribonuclease I (Invitrogen) according to the manufacturer's instructions. Gene specific cDNAs for the heavy and light chains were synthesized using the M-MLV Reverse Transcriptase, RNase H Minus, point mutant kit (Promega, cat. no. M3683) according to the manufacturer's instructions. The V_{H} and V_{L} genes were PCR-amplified using a Novagen-based Ig-primer set (Novagen, San Diego, Calif.) and Accuprime Pfx DNA polymerase (Invitrogen). All PCR products that matched the expected amplicon size of 500 bp were cloned into pCR4 TOPO vector (Invitrogen), and the constructs were transformed in Subcloning efficient DH5α competent cells (Invitrogen) according to the manufacturer's instructions.

Clones were screened by colony PCR using universal M13 forward and reverse primers, and at least two clones from each reaction were selected for DNA sequencing analysis. CDRs were identified following the Kabat rules (Kabat et al., 1991. Sequences of Proteins of Immunological Interest, Fifth Edition, NIH Publication No. 91-3242).

The sequences are disclosed in the Sequence Listing filed herewith and are listed above in Table 1.

The invention is further described by the following numbered paragraphs:

1. Method for obtaining CD70-binding peptides comprising:
providing a library of binder peptides;
selecting CD70-binding peptides from the library, by means of affinity selection using a target peptide immobilized on a solid support, said target peptide comprising a number of CD70 epitopes and a CD27-binding region of CD70;
characterized in that, the target peptide is in interaction with a peptide, the shielding peptide, comprising a CD70 binding region of CD27 or of a binding equivalent of CD27 capable of ligating CD70.

2. Method according to paragraph 1, wherein the library comprises a collection of lymphocytes, preferably splenocytes, collected from a mammal, such as a non-human mammal, immunized with an agent suitable for eliciting a CD70-specific immune response in the mammal.

3. Method according to any of the paragraphs 1-2, further comprising determining a peptide sequence of a selected CD70-binding peptide and/or a nucleotide sequence coding for a selected CD70-binding peptide.

4. CD70-binding peptide obtainable with a method according to any of the paragraphs 1-3.

5. CD70-binding peptide according to paragraph 4, comprising immunoglobulin V_{H} domains, comprising CDR1, CDR2 and CDR3 sequences having at least 60%, such as at least 85%, preferably at least 90%, more preferably at least 95% sequence similarity with amino acid sequences selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82.

6. CD70-binding peptide according to any of the paragraphs 4-5 comprising immunoglobulin V_{H} and V_{L} domains, comprising V_{H} CDR1, V_{H} CDR2, V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences selected from SEQ ID NO:3 and 4, or 13 and 14, or 23 and 24, or 33 and 34, or 43 and 44, or 53 and 54, or 63 and 64, or 73 and 74.

7. Cell comprising a nucleotide sequence coding for a CD70-binding peptide according to any of the paragraphs 5-6.

8. Process for producing a CD70-binding peptide comprising providing cells according to paragraph 7, culturing said cells and allowing the cells to express and preferably secrete the CD70-binding peptide.

9. CD70-binding peptide obtainable with the method according to paragraph 8.

10. CD70-binding peptide according to paragraph 9, comprising immunoglobulin V_{H} domains, comprising CDR1, CDR2 and CDR3 sequences having at least 60%, such as at least 85%, preferably at least 90%, more preferably at least 95% sequence similarity with amino acid sequences selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82.

11. CD70-binding peptide according to any of the paragraphs 9-10 comprising immunoglobulin V_{H} and V_{L} domains, comprising V_{H} CDR1, V_{H} CDR2 V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences selected from SEQ ID NO:3 and 4, or 13 and 14, or 23 and 24, or 33 and 34, or 43 and 44, or 53 and 54, or 63 and 64, or 73 and 74.

12. CD70-binding peptide according to any of the paragraphs 9-11 or 4-6 for use as a medicament, preferably a medicament for the treatment of cancer, more preferably a medicament for the treatment of a CD70 positive cancer, most preferably a CD70 over-expressing cancer.

13. Method for treating a CD70 positive cancer, most preferably a CD70 over-expressing cancer, comprising administering to a subject a therapeutically effective amount of a CD70-binding peptide according to any of the paragraphs 9-11 or 4-6.

14. Composition, such as a pharmaceutical composition, comprising a CD70-binding peptide according to any of the paragraphs 9-11 or 4-6, together with a carrier, such as a pharmaceutically acceptable carrier.

15. Use of a CD70-binding peptide according to any of the paragraphs 9-11 or 4-6 as a diagnostic tool, such as a diagnostic tool for use in an ex vivo diagnostic method, preferably in cancer diagnosis, more preferably in the diagnosis of a CD70 positive cancer, most preferably a CD70 over-expressing cancer, wherein in said use the CD70-binding peptide preferably is linked to a signaling moiety.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method for obtaining CD70-binding peptides comprising:
providing a library of binder peptides;
selecting CD70-binding peptides from the library, by means of affinity selection using a target peptide immobilized on a solid support, said target peptide comprising a number of CD70 epitopes and a CD27-binding region of CD70;
characterized in that, the target peptide is in interaction with a peptide, the shielding peptide, comprising a CD70 binding region of CD27 or of a binding equivalent of CD27 capable of ligating CD70.

2. The method according to claim 1, wherein the library comprises a collection of lymphocytes, preferably splenocytes, collected from a mammal, such as a non-human mammal, immunized with an agent suitable for eliciting a CD70-specific immune response in the mammal.

3. The method according to claim 1, further comprising determining a peptide sequence of a selected CD70-binding peptide and/or a nucleotide sequence coding for a selected CD70-binding peptide.

4. A CD70-binding peptide obtainable with a method according to claim 1.

5. The CD70-binding peptide according to claim 4, comprising immunoglobulin V_{H} domains, comprising CDR1, CDR2 and CDR3 sequences having at least 60%, such as at least 85%, preferably at least 90%, more preferably at least 95% sequence similarity with amino acid sequences selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82.

6. The CD70-binding peptide according to claim 4 comprising immunoglobulin V_{H} and V_{L} domains, comprising V_{H} CDR1, V_{H} CDR2, V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences selected from SEQ ID NO:3 and 4, or 13 and 14, or 23 and 24, or 33 and 34, or 43 and 44, or 53 and 54, or 63 and 64, or 73 and 74.

7. An anti-CD70 antibody, or antigen-binding fragment thereof, comprising immunoglobulin V_{H} domains, comprising CDR1, CDR2 and CDR3 sequences having at least 85%, preferably at least 90%, more preferably at least 95% sequence similarity with amino acid sequences selected from SEQ ID NO: 5, 6 and 7, or SEQ ID NO: 15, 16 and 17, or SEQ ID NO: 25, 26 and 27, or SEQ ID NO: 35, 36 and 37, or SEQ ID NO: 45, 46 and 47, or SEQ ID NO: 55, 56 and 57, or SEQ ID NO: 65, 66 and 67, or SEQ ID NO: 75, 76 and 77, or SEQ ID NO: 83, 84 and 85.

8. The anti-CD70 antibody, or antigen-binding fragment thereof, of claim 7 comprising immunoglobulin V_{H} and V_{L} domains, comprising V_{H} CDR1, V_{H} CDR2, V_{H} CDR3, V_{L} CDR1, V_{L} CDR2 and V_{L} CDR3 sequences having at least 85%, preferably at least 90%, more preferably at least 95% sequence similarity with amino acid sequences selected from SEQ ID NO: 5, 6, 7, 8, 9 and 10, or SEQ ID NO: 15, 16, 17, 18, 19 and 20, or SEQ ID NO: 25, 26, 27, 28, 29 and 30, or SEQ ID NO: 35, 36, 37, 38, 39 and 40, or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80.

9. The anti-CD70 antibody, or antigen-binding fragment thereof, of claim 7 which retains at least 80% or more of its respective parental antibody's binding affinity for CD70, said respective parental antibody being an antibody having as a V_{H} and V_{L} domain pair amino acid sequences selected from SEQ ID NO:3 and 4, or 13 and 14, or 23 and 24, or 33 and 34, or 43 and 44, or 53 and 54, or 63 and 64, or 73 and 74.

10. The anti-CD70 antibody, or antigen-binding fragment thereof, of claim 7 which is a humanized antibody, or antigen-binding fragment thereof.

11. A cell comprising a nucleotide sequence coding for a CD70-binding peptide or for an anti-CD70 antibody, or antigen-binding fragment thereof, according to claim 5, 6 or 7.

12. A process for producing a CD70-binding peptide or an anti-CD70 antibody, or antigen-binding fragment thereof, comprising providing cells according to claim 11, culturing said cells and allowing the cells to express and preferably secrete the CD70-binding peptide or the anti-CD70 antibody.

13. A CD70-binding peptide or an anti-CD70 antibody, or antigen-binding fragment thereof, obtainable with the method according to claim 12.

14. The CD70-binding peptide or the anti-CD70 antibody, or antigen-binding fragment thereof, according to claim 13, comprising immunoglobulin V_{H} domains, comprising CDR1, CDR2 and CDR3 sequences having at least 60%, such as at least 85%, preferably at least 90%, more preferably at least 95% sequence similarity with amino acid sequences selected from SEQ ID NO: 5, 6 and 7, or SEQ ID NO: 15, 16 and 17 or SEQ ID NO: 25, 26 and 27 or SEQ ID NO: 35, 36 and 37 or SEQ ID NO: 45, 46 and 47 or SEQ ID NO: 55, 56 and 57 or SEQ ID NO: 65, 66 and 67 or, SEQ ID NO: 75, 76 and 77 or SEQ ID NO: 83, 84 and 85, such as a V_{H} domain having at least 60%, such as at least 85%, preferably at least 90%, more preferably at least 95% sequence similarity with an amino acid sequence selected from SEQ ID NO. 3, 13, 23, 33, 43, 53, 63, 73 or 82.

15. The CD70-binding peptide or the anti-CD70 antibody, or antigen-binding fragment thereof, according to claim 13 comprising immunoglobulin V_{H} and V_{L} domains, comprising V_{H} CDR1, V_{H} CDR2 V_{H} CDR3, V_{L} CDR1, V_{L} CDR2 and V_{L} CDR3 sequences having at least 60%, such as at least 85%, preferably at least 90%, more preferably at least 95% sequence similarity with amino acid sequences selected from SEQ ID NO: 5, 6, 7, 8, 9 and 10 or SEQ ID NO: 15, 16, 17, 18, 19 and 20 or SEQ ID NO: 25, 26, 27, 28, 29 and 30 or SEQ ID NO: 35, 36, 37, 38, 39 and 40 or SEQ ID NO: 45, 46, 47, 48, 49 and 50, or SEQ ID NO: 55, 56, 57, 58, 59 and 60, or SEQ ID NO: 65, 66, 67, 68, 69 and 70, or SEQ ID NO: 75, 76, 77, 78, 79 and 80, such as a V_{H} and V_{L} domain pair having at least 60%, such as at least 85%, preferably at least 90%, more preferably at least 95% sequence similarity with amino acid sequences selected from SEQ ID NO:3 and 4, or 13 and 14, or 23 and 24, or 33 and 34, or 43 and 44, or 53 and 54, or 63 and 64, or 73 and 74.

16. A CD70-binding peptide or an anti-CD70 antibody, or antigen-binding fragment thereof, according to claim 4 for use as a medicament, preferably a medicament for the treatment of cancer, more preferably a medicament for the treatment of a CD70 positive cancer, most preferably a CD70 over-expressing cancer.

17. A CD70-binding peptide or an anti-CD70 antibody, or antigen-binding fragment thereof, according to claim 13 for use as a medicament, preferably a medicament for the treatment of cancer, more preferably a medicament for the treatment of a CD70 positive cancer, most preferably a CD70 over-expressing cancer.

18. A method for treating a CD70 positive cancer, most preferably a CD70 over-expressing cancer, comprising administering to a subject a therapeutically effective amount of a CD70-binding peptide or an anti-CD70 antibody, or antigen-binding fragment thereof, according to claim 4.

19. A method for treating a CD70 positive cancer, most preferably a CD70 over-expressing cancer, comprising administering to a subject a therapeutically effective amount of a CD70-binding peptide or an anti-CD70 antibody, or antigen-binding fragment thereof, according to claim 13.

20. A composition, such as a pharmaceutical composition, comprising a CD70-binding peptide or an anti-CD70 antibody, or antigen-binding fragment thereof, according to claim 4, together with a carrier, such as a pharmaceutically acceptable carrier.

21. A composition, such as a pharmaceutical composition, comprising a CD70-binding peptide or an anti-CD70 antibody, or antigen-binding fragment thereof, according to claim 13, together with a carrier, such as a pharmaceutically acceptable carrier.
